(19) United States
(12) Patent Application Publication
(10) Pub. No.: US 2025/0320520 A1
(43) Pub. Date: Oct. 16, 2025

(54) TRANGENIC RODENTS FOR CELL LINE IDENTIFICATION AND ENRICHMENT

(71) Applicant: ABCELLERA BIOLOGICS INC., Vancouver, British Columbia (CA)

(72) Inventors: Ping XIANG, Vancouver, British Columbia (CA); Wei WEI, Vancouver, British Columbia (CA); Davide PELLACANI, Vancouver, British Columbia (CA); Jens RUSCHMANN, Vancouver, British Columbia (CA)

(21) Appl. No.: 18/695,100

(22) PCT Filed: Sep. 30, 2022
(86) PCT No.: PCT/US2022/077363

§ 371 Date: Mar. 25, 2024

Related U.S. Application Data
(60) Provisional application No. 63/261,987, filed on Oct. 1, 2021.

(51) Int. Cl. C12N 15/86; A01K 67/0278; C07K 16/00; C12N 5/10; C12N 9/22; C12N 15/113; C12N 15/65; G01N 33/533

(52) U.S. Cl. CPC C12N 15/86; A01K 67/0278; C07K 16/00; C12N 5/10; C12N 9/222; C12N 15/113; C12N 15/65; G01N 33/533; A01K 2217/072; A01K 2227/105; C12N 2510/04

(57) ABSTRACT

The disclosure provides nucleic acid constructs comprising a transmembrane reporter cassette encoding an affinity tag, a transmembrane (TM) domain and a fluorescent reporter protein. In embodiments, the nucleic acid constructs are inserted in a safe harbor locus or an immunoglobulin constant domain locus of in a cell of a non-human mammal. In embodiments, when the transmembrane reporter cassette is expressed in the cell, the affinity tag is displayed on a surface of the cell while the fluorescent reporter protein is located inside the cell membrane. The presence of the affinity tag and the fluorescent reporter protein allow for identification, sorting and/or isolation of cells expressing the nucleic acid constructs. The disclosure also provides embodiments of methods of modifying cells and non-human organisms with the nucleic acid constructs, along with embodiments of cells and non-human organisms produced using the disclosed methods.

### FIELD OF THE INVENTION

The present disclosure relates to nucleic acid constructs, transgenic rodents, rodent cell lines, and methods that allow for identification and enrichment of specific cell types, for instance, of cells in a specific stage of development, of cells expressing a specific promoter, or of cells expressing specific proteins such as antibodies.

### BACKGROUND OF THE INVENTION

Identifying and enriching cells engineered to express a specific protein or in a specific stage of development is a key challenge in the development of biological therapeutics. To enrich for specific cell populations the common workflow is to generate a single cell suspension, stain the cell mixture with a panel of antibodies recognizing surface markers, and then separate the cells using either magnetic- or flow-based methods. However, this procedure is limited by current knowledge of cell type specific cell-surface markers and the specificity and availability of antibodies to recognize those markers. The procedure generally results in less than ideal yield and purity of cells of interest following enrichment, with a high proportion of unwanted contaminating cells and a loss of cells of interest during enrichment. For example, common strategies to identify Ig expressing cells are based on known endogenous lineage surface markers combined with antibody staining and detection of those markers.

Commonly used antibodies to enrich for mouse Ig expressing cells are anti-CD19, anti-CD138, and anti-Ig antibodies. However, differential expression of these three markers during B-cell differentiation means not all populations can be efficiently enriched using cell surface markers. For example, CD19 is considered a pan-B cell marker (including B cell progenitors that do not express Ig) but its expression is decreased dramatically in antibody secreting cells and therefore it cannot enrich that valuable population. CD138 is considered a plasma cell marker, but is also expressed in some early stage progenitor B cells that do not express Ig. This marker will therefore enrich this unwanted population. During B cell development, after pre B cells differentiate into immature B cells, they start to display Ig on their cell surface, therefore this population can be captured using the Ig marker. However, after mature B cells fully differentiate into plasma cells, Ig surface expression is lost. As a consequence, when using these markers to enrich Ig expressing cells with magnetic-based strategies (which provides better scale and time efficiency compared to flow-based sorting), the resulting enriched cell populations often include contaminants of non-Ig expressing B cells, with inefficient enrichment and loss of antibody secreting cells.

Isolation and enrichment of cell lines that express tissue specific promoters is also a challenge for similar reasons. Tissue specificity is largely determined by transcription factors, meaning that cell surface markers may not be available for enrichment of cell lines expressing a protein in a tissue specific manner, or the available markers may not be specific enough to provide useful enrichment.

### SUMMARY OF THE INVENTION

In embodiments, the present disclosure provides a nucleic acid construct comprising a leader sequence, a LoxP-Stop-LoxP cassette, and a transmembrane reporter cassette encoding an affinity tag, a transmembrane (TM) domain and a fluorescent reporter protein. In embodiments, the nucleic acid construct comprises single stranded DNA, double stranded DNA, a plasmid, or a viral vector.

In embodiments, the nucleic acid construct further comprises a first homology arm and a second homology arm that are homologous to a first target sequence and a second target sequence, respectively, within a safe harbor locus in a non-human mammal. In embodiments, the first homology and second homology arms, each independently, comprise from about 15 nucleotides to about 12000 nucleotides.

In embodiments of the nucleic acid construct, the safe harbor locus comprises a Rosa26 locus on chromosome 6 in a genome of a mouse or a Hipp11 locus on chromosome 11 in a genome of a mouse.

In embodiments, the nucleic acid construct further comprises a promoter. In embodiments, the promoter comprises a mammalian promoter. In embodiments, the promoter comprises a CAG, CMV, EF1a, SV40, PGK1, Ubc or human beta actin promoter. In embodiments, the leader sequence comprises a secretory signal peptide. In embodiments, the secretory signal peptide comprises the IL-2 leader sequence MYRMQLLSCIALSLALVINS (SEQ ID NO:2).

In embodiments of the nucleic acid construct, the affinity tag comprises a StrepII-tag. In embodiments, the affinity tag comprises tandem repeats of a StrepII-tag. In embodiments, the affinity tag comprises from about 1 to about 18 tandem repeats of a StrepII-tag with a tag linker in between repeats. In embodiments, the affinity tag comprises 3 tandem repeats of a StrepII-tag. In embodiments, the StrepII-tag comprises an eight amino acid peptide sequence of WSHPQFEK (SEQ ID NO: 1). In embodiments, the transmembrane domain comprises a hydrophobic α-helix.

In embodiments of the nucleic acid construct, the fluorescent reporter protein comprises green fluorescent protein (GFP), enhanced green fluorescent protein (EGFP), enhanced yellow fluorescent protein (EYFP) or enhanced cyan fluorescent protein (ECFP).

In embodiments, the present disclosure provides a method of generating a genetically modified non-human mammal cell, the method comprising: (a) introducing a nucleic acid construct described herein into the non-human mammal cell; and (b) introducing a nuclease into the non-human mammal cell, wherein the nuclease causes a single strand break or a double strand break at a safe harbor locus in a genome of the non-human mammal cell, wherein the nucleic acid construct is integrated into the genome of the non-human mammal cell at the safe harbor locus by homologous recombination.

In embodiments of the method, the introducing the nuclease comprises introducing an expression construct encoding the nuclease. In embodiments, introducing the nuclease comprises introducing a mRNA encoding the nuclease. In embodiments, the nuclease comprises a Zinc Finger nuclease (ZFN), a transcription activator-Like Effector Nuclease (TALEN), a Meganuclease, or a Clustered Regularly Interspaced Short Palindromic Repeats (CRISPR)-associated (Cas) protein and a guide RNA (gRNA). In embodiments, the gRNA comprises a CRISPR RNA (crRNA) that targets a recognition site and a trans-activating CRISPR RNA (tracrRNA). In embodiments, the CRISPR-Cas protein comprises Cas9.

In embodiments of the method, the non-human mammal cell is a rodent cell. In embodiments, the rodent cell is a rat cell or a mouse cell. In embodiments, the safe harbor locus comprises a Rosa26 locus on chromosome 6 or a Hipp11 locus on chromosome 11 in a genome of a mouse. In embodiments, the non-human mammal cell is a pluripotent cell. In embodiments, the pluripotent cell is a non-human zygote or a non-human embryonic stem (ES) cell. In embodiments, the pluripotent cell is a mouse zygote cell or rat zygote cell. In embodiments, the pluripotent cell is a mouse embryonic stem (ES) cell or rat embryonic stem (ES) cell.

In embodiments, the method further comprises isolating the genetically modified non-human mammal cell in which the nucleic acid construct is integrated at the safe harbor locus.

In embodiments, the present disclosure provides a genetically modified a non-human mammal cell generated by a method of generating a genetically modified non-human mammal cell described herein.

In embodiments of the method, the method further comprises injecting the isolated cell into a blastocyst and generating a transgenic non-human mammal comprising the nucleic acid construct integrated into the safe harbor locus. In embodiments, the disclosure provides a genetically modified non-human transgenic mammal generated by this method. In embodiments, the mammal is a rodent. In embodiments, the rodent is a rat or a mouse.

In embodiments, the method further comprises breeding the transgenic non-human mammal comprising the nucleic acid construct integrated into the safe harbor locus with a transgenic non-human mammal that expresses Cre recombinase to obtain a non-human mammal with cells that express a fusion protein comprising an affinity tag, a transmembrane domain and a fluorescent reporter protein. In embodiments, the transgenic non-human mammal comprising the nucleic acid construct integrated into the safe harbor locus is a mouse comprising the nucleic acid construct integrated into a Rosa26 locus and the transgenic non-human mammal that expresses Cre recombinase is a mouse. In embodiments, the transgenic non-human mammal comprising the nucleic acid construct integrated into the safe harbor locus is a mouse comprising the nucleic acid construct integrated into a Hipp11 locus and the transgenic non-human mammal that expresses Cre recombinase is a mouse. In embodiments, Cre expression in the transgenic mouse is tissue specific. In embodiments, the present disclosure provides a genetically modified non-human mammal with cells that express a fusion protein comprising an affinity tag, a transmembrane domain and a fluorescent reporter protein generated by this method.

In embodiments, the present disclosure provides a genetically modified non-human mammal cell comprising a genome comprising a nucleic acid construct described herein integrated into a safe harbor locus. In embodiments, the safe harbor locus comprises a Rosa26 locus on chromosome 26 in a genome of a mouse or a Hipp11 locus on chromosome 11 in a genome of a mouse. In embodiments, the genetically modified non-human mammal cell is a hybridoma or an immortalized cell.

In embodiments of the genetically modified non-human mammal cell, the cell expresses a fusion protein comprising an affinity tag, a transmembrane domain and a fluorescent reporter protein. In embodiments, the affinity tag is expressed on a cell surface of the non-human mammal cell. In embodiments, the affinity tag comprises a StrepII-tag. In embodiments, the fluorescent reporter protein is exposed on a cytosolic surface of the non-human mammal cell. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP).

In embodiments, the present disclosure provides a method for isolating cells obtained from a genetically modified non-human mammal, the method comprising: (a) obtaining cells from a genetically modified non-human mammal described herein; (b) screening the cells obtained from the genetically modified non-human mammal for expression of a fusion protein comprising an affinity tag, a transmembrane domain and a fluorescent reporter protein; and (c) isolating cells expressing the fusion protein.

In embodiments of the method for isolating cells, the cells are screened by fluorescent activated cell sorting (FACS) or magnetic activated cell sorting (MACS). In embodiments, the affinity tag is expressed on a cell surface of the genetically modified non-human mammal cell. In embodiments, the affinity tag comprises a StrepII-tag. In embodiments, the fluorescent reporter protein is exposed on a cytosolic surface of the non-human mammal cell. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP).

In embodiments, the present disclosure further provides a nucleic acid construct comprising a linker, a leader sequence, and a transmembrane reporter cassette encoding an affinity tag, a transmembrane domain and a fluorescent reporter.

In embodiments, the nucleic acid construct comprises single stranded DNA, double stranded DNA, a plasmid, or a viral vector. In embodiments, the nucleic acid construct further comprises a first homology arm and a second homology arm that are homologous to a first target sequence and a second target sequence, respectively. In embodiments, the first target sequence is upstream of an immunoglobulin constant domain locus and the second target sequence is downstream of a stop codon of the immunoglobulin constant domain locus. In embodiments, the immunoglobulin constant domain locus is an immunoglobulin light chain constant domain locus. In embodiments, the immunoglobulin light chain constant domain locus is an immunoglobulin kappa constant domain locus. In embodiments, the immunoglobulin light chain constant domain locus is an immunoglobulin lambda constant domain locus. In embodiments, the immunoglobulin constant domain locus is an immunoglobulin heavy chain constant domain locus. In embodiments, the immunoglobulin heavy chain constant domain locus is a gamma, delta, alpha, mu or epsilon immunoglobulin heavy chain constant domain locus.

In embodiments of the nucleic acid construct, the first homology and second homology arms, each independently, comprise from about 15 nucleotides to about 12000 nucleotides. In embodiments, the linker comprises a stop codon and an Internal Ribosomal Entry Site (IRES). In embodiments, the linker comprises a protease recognition site and a self-cleaving peptide. In embodiments, the linker comprises a leaky stop codon (LSC) with a peptide linker, a protease recognition site, and a self-cleaving peptide. In embodiments, the protease recognition site comprises a Furin protease recognition site. In embodiments, the Furin protease recognition site comprises a nucleic acid sequence encoding the peptide of Arg-X-Arg-Arg. In embodiments, X is a hydrophobic amino acid. In embodiments, X is a hydrophilic amino acid. In embodiments, X is lysine. In embodiments, the Furin protease recognition site comprises a nucleic acid sequence encoding the peptide of X-Arg-X-Lys-Arg-X or X-Arg-X-Arg-Arg-X. In embodiments, X is a hydrophobic amino acid. In embodiments, the hydrophobic amino acid is Gly, Ala, Ile, Leu, Met, Val, Phe, Trp or Tyr. In embodiments, X is a hydrophilic amino acid. In embodiments, the hydrophilic amino acid is lysine. In embodiments, the self-cleaving peptide comprises a 2A self-cleaving peptide. In embodiments, the leaky stop codon comprises TGACTAG. In embodiments, the dipeptide linker comprises Leu-Gly.

In embodiments of the nucleic acid construct, the leader sequence comprises a secretory signal peptide. In embodiments, the secretory signal peptide comprises the IL-2 leader sequence MYRMQLLSCIALSLALVTNS (SEQ ID NO: 2).

In embodiments of the nucleic acid construct, the affinity tag comprises a StrepII-tag. In embodiments, the affinity tag comprises tandem repeats of a StrepII-tag. In embodiments, the affinity tag comprises from about 1 to about 18 tandem repeats of a StrepII-tag with a tag linker in between repeats. In embodiments, the affinity tag comprises 3 tandem repeats of a StrepII-tag. In embodiments, the StrepII-tag comprises an eight amino acid peptide sequence of WSHPQFEK (SEQ ID NO: 1). In embodiments, the transmembrane domain comprises a hydrophobic α-helix.

In embodiments of the nucleic acid construct, the fluorescent reporter protein comprises green fluorescent protein (GFP), enhanced green fluorescent protein (EGFP), enhanced yellow fluorescent protein (EYFP) or enhanced cyan fluorescent protein (ECFP).

In embodiments, the present disclosure provides a method of generating a genetically modified non-human mammalian cell, the method comprising: (a) introducing a nucleic acid construct described herein into the non-human mammal cell; and (b) introducing a nuclease into the non-human mammal cell, wherein the nuclease causes a single strand break or a double strand break at an immunoglobulin constant domain locus in a genome of the non-human mammal cell, and the nucleic acid construct is integrated into the genome of the non-human mammal cell at the immunoglobulin constant domain locus by homologous recombination. In embodiments, the immunoglobulin constant domain locus is an immunoglobulin light chain constant domain locus. In embodiments, the immunoglobulin light chain constant domain locus is a kappa light chain constant domain locus. In embodiments, the immunoglobulin light chain constant domain locus is a lambda light chain constant domain locus. In embodiments, the immunoglobulin constant domain locus is an immunoglobulin heavy chain constant domain locus. In embodiments, the immunoglobulin heavy chain constant domain locus is a gamma, delta, alpha, mu or epsilon immunoglobulin constant domain locus.

In embodiments of the method, introducing the nuclease comprises introducing an expression construct encoding the nuclease. In embodiments, introducing the nuclease comprises introducing a mRNA encoding the nuclease. In embodiments, the nuclease comprises a Zinc Finger nuclease (ZFN), a transcription activator-Like Effector Nuclease (TALEN), a Meganuclease, or a Clustered Regularly Interspaced Short Palindromic Repeats (CRISPR)-associated (Cas) protein and a guide RNA (gRNA). In embodiments, the gRNA comprises a CRISPR RNA (crRNA) that targets a recognition site and a trans-activating CRISPR RNA (tracrRNA). In embodiments, the CRISPR-Cas protein comprises Cas9.

In embodiments of the method, the non-human mammal cell is a rodent cell. In embodiments, the rodent cell is a rat cell or a mouse cell. In embodiments, the non-human mammal cell is a pluripotent cell. In embodiments, the pluripotent cell is a non-human embryonic stem (ES) cell. In embodiments, the pluripotent cell is a mouse embryonic stem (ES) cell or rat embryonic stem (ES) cell.

In embodiments, the method further comprises isolating the genetically modified non-human mammal cell in which the nucleic acid construct is integrated at an immunoglobulin constant domain locus. In embodiments, the immunoglobulin constant domain locus is an immunoglobulin light chain constant domain locus. In embodiments, the immunoglobulin light chain constant domain locus is a kappa light chain constant domain locus. In embodiments, the immunoglobulin light chain constant domain locus is a lambda light chain constant domain locus. In embodiments, the immunoglobulin constant domain locus is an immunoglobulin heavy chain constant domain locus. In embodiments, the immunoglobulin heavy chain constant domain locus is a gamma, delta, alpha, mu or epsilon immunoglobulin constant domain locus.

In embodiments, the present disclosure provides a genetically modified a non-human mammal cell generated by a method disclosed herein.

In embodiments, the method further comprises injecting the isolated cell into a blastocyst and generating a transgenic non-human mammal comprising the nucleic acid construct integrated into the immunoglobulin constant domain locus. In embodiments, the immunoglobulin constant domain locus is an immunoglobulin light chain constant domain locus. In embodiments, the immunoglobulin light chain constant domain locus is a kappa light chain constant domain locus. In embodiments, the immunoglobulin light chain constant domain locus is a lambda light chain constant domain locus. In embodiments, the immunoglobulin constant domain locus is an immunoglobulin heavy chain constant domain locus. In embodiments, the immunoglobulin heavy chain constant domain locus is a gamma, delta, alpha, mu or epsilon immunoglobulin constant domain locus. In embodiments, the present disclosure provides a genetically modified non-human transgenic mammal generated by this method.

In embodiments, the present disclosure provides a genetically modified non-human mammal cell comprising a genome comprising a nucleic acid construct described herein integrated into an immunoglobulin constant domain locus. In embodiments, the genetically modified non-human mammal cell comprises a genome comprising a nucleic acid construct described herein integrated into an immunoglobulin constant domain locus. In embodiments, the immunoglobulin constant domain locus is a light chain constant domain locus. In embodiments, the light chain constant domain locus is a kappa constant domain locus. In embodiments, the light chain constant domain locus is a lambda constant domain locus. In embodiments, the constant domain locus is a heavy chain constant domain locus. In embodiments, the immunoglobulin expressing cell is obtained from an immunized mammal. In embodiments, the cell is an immunoglobulin expressing cell. In embodiments, the genetically modified non-human mammal cell expresses an immunoglobulin kappa light chain.

In embodiments of the immunoglobulin expressing non-human mammal cell, the cell is an immature B cells or a descendant of an immature B cell. In embodiments, the cell is a hybridoma, a stem cell or an immortalized cell.

In embodiments of the immunoglobulin expressing non-human mammal cell, the cell expresses a fusion protein comprising an affinity tag, a transmembrane domain and a fluorescent reporter protein. In embodiments, the affinity tag is expressed on a cell surface of the non-human mammal cell. In embodiments, the affinity tag comprises a StrepII-tag. In embodiments, the fluorescent reporter protein is exposed on a cytosolic surface of the non-human mammal cell. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP). In embodiments, the fluorescent reporter protein comprises red fluorescent protein (RFP). In embodiments, the red fluorescent protein is monomeric cherry (mCherry) or tandem dimer Tomato (tdTomato). Other fluorescent proteins are known and can be used in the construct described herein. See, for example, Li et al. (2018) “Overview of the reporter genes and reporter mouse models,” Anim Models and Exp Med. 1:29-35 (doi.org/10.1002/ame2.12008).

In embodiments of the immunoglobulin expressing non-human mammal cell, expression of the fusion protein is driven by an endogenous immunoglobulin transcription regulator. In embodiments, the endogenous immunoglobulin transcription regulator is an endogenous immunoglobulin light chain transcription regulator. In embodiments, the endogenous immunoglobulin light chain transcription regulator comprises a promoter, and other cis-regulatory elements in the mouse light chain locus. In embodiments, the endogenous immunoglobulin kappa light chain transcription regulator comprises a promoter, and other cis-regulatory elements in the mouse light chain locus. In embodiments, the endogenous immunoglobulin lambda light chain transcription regulator comprises a promoter, and other cis-regulatory elements in the mouse light chain locus. In embodiments, the endogenous immunoglobulin transcription regulator is an endogenous immunoglobulin heavy chain transcription regulator. In embodiments, the endogenous immunoglobulin heavy light chain transcription regulator comprises a promoter, and other cis-regulatory elements in the mouse heavy chain locus.

In embodiments, the present disclosure provides a method for identifying immunoglobulin expressing cells obtained from a genetically modified non-human mammal, the method comprising: (a) obtaining cells from a genetically modified non-human mammal described herein; (b) screening the cells obtained from the genetically modified non-human mammal for expression of a fusion protein comprising an affinity tag, a transmembrane domain and a fluorescent reporter protein; and (c) identifying immunoglobulin expressing cells based on expression of the fusion protein.

In embodiments of the method, the cells are screened by fluorescent activated cell sorting (FACS) or magnetic activated cell sorting (MACS). In embodiments, the affinity tag is expressed on a cell surface of the genetically modified non-human mammal cell. In embodiments, the affinity tag comprises a StrepII-tag. In embodiments, the fluorescent reporter protein is exposed on a cytosolic surface of the non-human mammal cell. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP). In embodiments, the fluorescent reporter protein comprises red fluorescent protein (RFP). In embodiments, the red fluorescent protein is monomeric cherry (mCherry) or tandem dimer Tomato (tdTomato).

In embodiments of the method, the genetically modified non-human mammal has been immunized with an antigen of interest. In embodiments, the immunoglobulin expressing cells express an immunoglobulin light chain. In embodiments, the immunoglobulin expressing cells express an immunoglobulin kappa light chain. In embodiments, the immunoglobulin expressing cells express an immunoglobulin lambda light chain. In embodiments, the immunoglobulin expressing cells express an immunoglobulin heavy chain. In embodiments, the immunoglobulin expressing cells comprise immature B cells and their descendants.

In embodiments, the method further comprises isolating an immunoglobulin expressed from the cell obtained from a genetically modified non-human mammal. In embodiments, the present disclosure provides an immunoglobulin obtained by this method.

In embodiments, the present disclosure provides a method of producing a therapeutic or diagnostic immunoglobulin, the method comprising: (i) cloning a variable domain of an immunoglobulin described herein; and (ii) generating the therapeutic or diagnostic immunoglobulin comprising the variable domain obtained in (i).

In embodiments, the present disclosure provides a method of producing a monoclonal antibody, the method comprising: (i) obtaining immunoglobulin expressing cells from a genetically modified non-human mammal described herein; (ii) immortalizing the immunoglobulin expressing cells obtained in (i); and (iii) isolating monoclonal antibodies expressed by the immortalized immunoglobulin expressing cells, or nucleic acid sequences encoding the monoclonal antibodies. In embodiments, the method further comprises: (iv) cloning a variable domain of the isolated monoclonal antibody; and (v) producing a therapeutic or diagnostic antibody comprising the cloned variable domain. In embodiments, the present disclosure provides a therapeutic or diagnostic antibody produced by this method.

### BRIEF DESCRIPTION OF THE FIGURES

FIG. 1A-C are schematics of the construction and use of an embodiment of a conditional reporter nucleic acid construct as described herein. As shown in FIG. 1A, a nucleic acid construct is inserted in the ROSA26 locus safe harbor site. In the figure, CAGGS represents a CAG promoter, L represents a Leader sequence, the LoxP-Stop-LoxP cassette arranged as shown, STX3 represents a three tandem repeats of the Strep-II tag, TM represents a transmembrane domain and GFP represents a green fluorescent protein reporter. FIG. 1B is a schematic of the cross performed with a Cre switch line and the conditional reporter line to form a tissue specific reporter mouse line. As shown schematically, after conditional reporter line is bred with switch line, the cre recombinase removes the stop codon in front of the reporter to switch on expression of the reporter within the nucleus of cre expressing cells. As a result, these cells are permanently labeled with affinity tag on the cell surface and the intracellular fluorescence marker. FIG. 1C is a schematic representation of how cells isolated from the switch reporter line represented in FIG. 1B are separated using FACS or MACS as described herein.

FIG. 2 is a schematic of the targeting strategy of Mouse/Rat IgK locus. After targeting, the labelling cassette is knocked in at the stop codon of the IgK gene and under the control of IgK locus promoter (note that LK in the figure below is a linker sequence, details in following sections). In the figure, the black rectangles represent the V and J segments of the region; LK represents a linker sequence; L represents a leader sequence, STX3 represents a three tandem repeats of the Strep-II tag, TM represents a transmembrane domain, and GFP represents a green fluorescent protein reporter.

FIG. 3 is a schematic of the formation of an embodiments of an IgK reporter mouse formed as described herein, and a schematic of how pooled cells isolated from the mouse are separated using FACS or MACS as described herein.

### DETAILED DESCRIPTION OF THE INVENTION

The present disclosure provides embodiments of nucleic acid constructs comprising a transmembrane reporter cassette encoding an affinity tag, a transmembrane (TM) domain and a fluorescent reporter protein. In embodiments, the nucleic acid constructs are inserted in a safe harbor locus or an immunoglobulin constant domain locus of in a cell of a non-human mammal. In embodiments, when the transmembrane reporter cassette is expressed in the cell, the affinity tag is displayed on a surface of the cell and the fluorescent reporter protein is located inside the cell membrane. The presence of the affinity tag and the fluorescent reporter protein allow for identification, sorting and/or isolation of cells expressing the nucleic acid constructs. The present disclosure also provides embodiments of methods of modifying cells and non-human organisms with the nucleic acid constructs, along with embodiments of cells and non-human organisms produced using the disclosed methods.

### A. Definitions

Unless otherwise defined, scientific and technical terms used herein shall have the meanings that are commonly understood by those of ordinary skill in the art. Further, unless otherwise required by context, singular terms shall include pluralities and plural terms shall include the singular, for example, “a” or “an”, include pluralities, e.g., “one or more” or “at least one” and the term “or” can mean “and/or”, unless stated otherwise. The terms “including”, “includes” and “included”, are not limiting. Ranges provided herein, of any type, include all values within a particular range described and values about an endpoint for a particular range.

As used herein, the term “about” is used to modify, for example, the quantity of an ingredient in a composition, concentration, volume, process temperature, process time, yield, flow rate, pressure, and ranges thereof, employed in describing the invention. The term “about” refers to variation in the numerical quantity that can occur, for example, through typical measuring and handling procedures used for making compounds, compositions, concentrates or formulations; through inadvertent error in these procedures; through differences in the manufacture, source, or purity of starting materials or ingredients used to carry out the methods, and other similar considerations. The term “about” also encompasses amounts that differ due to aging of a formulation with a particular initial concentration or mixture, and amounts that differ due to mixing or processing a formulation with a particular initial concentration or mixture. Where modified by the term “about,” the claims appended hereto include such equivalents.

Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “polypeptide” or “protein” can be used interchangeably to refer to a molecule having two or more amino acid residues joined to each other by peptide bonds. The term “polypeptide” can refer to antibodies and other non-antibody proteins. Non-antibody proteins include, but are not limited to, proteins such as enzymes, receptors, ligands of a cell surface protein, secreted proteins and fusion proteins or fragments thereof. Polypeptides can be of scientific or commercial interest, including protein-based therapeutics.

As used herein, the terms “antibody” and “immunoglobulin” can be used interchangeably and refer to a polypeptide or group of polypeptides that include at least one binding domain that is formed from the folding of polypeptide chains having three-dimensional binding spaces with internal surface shapes and charge distributions complementary to the features of an antigenic determinant of an antigen. Naturally-occurring antibodies typically have a tetrameric form, with two pairs of polypeptide chains, each pair having one “light” and one “heavy” chain. The variable regions of each light/heavy chain pair form an antibody binding site. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains (CH). Each light chain has a variable domain at one end (VL) and a constant domain (CL) at its other end, wherein the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. Heavy chains are classified as either gamma chains, delta chains, alpha chains, mu chains or epsilon chains based on the amino acid sequence of the heavy chain constant region.

The terms “antigen-binding fragment” or “immunologically active fragments” refer to fragments of an antibody that contain at least one antigen-binding site and retain the ability to specifically bind to an antigen. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., Glm (f, z, a or x), G2m (n), G3m (g, b, or c), Am, Em, and Km (1, 2 or 3)). Subisotypes can include subclasses such as those found in non-human mammals such as rodents, for example IgG1, IgG2a, IgG2b, IgG2c and IgG3. Immunoglobulins include, but are not limited to, monoclonal antibodies (including full-length monoclonal antibodies), polyclonal antibodies, multispecific antibodies formed from at least two different epitope binding fragments (e.g., bispecific antibodies), CDR-grafted, human antibodies, humanized antibodies, camelized antibodies, chimeric antibodies, anti-idiotypic (anti-Id) antibodies, intrabodies, and desirable antigen binding fragments thereof, including recombinantly produced antibody fragments. Examples of antibody fragments that can be recombinantly produced include, but are not limited to, antibody fragments that include variable heavy- and light-chain domains, such as single-chain Fvs (scFv), single-chain antibodies, Fab fragments, Fab′ fragments, F(ab′)₂ fragments. Antibody fragments can also include epitope-binding fragments or derivatives of any of the antibodies enumerated above.

The term “recombinant” refers to a biological material, for example, a nucleic acid or protein, that has been artificially or synthetically (i.e., non-naturally) altered or produced by human intervention. The term “recombinant antibody” refers to an antibody prepared by recombinant DNA processes, including, for example, antibodies expressed using a recombinant expression vector transfected into a host cell, as well as antibodies isolated from a recombinant, combinatorial human antibody library. In embodiments, the recombinant antibody is a recombinant human antibody, which includes, but is not limited to, antibodies isolated from a transgenic animal having human immunoglobulin genes or antibodies prepared by splicing a human immunoglobulin gene sequences into another DNA sequence.

A “coding sequence” or a sequence which “encodes” a selected polypeptide, as used herein, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” as used herein refers to a nucleic acid sequence which codes for a polypeptide sequence, wherein the polypeptide sequence or a portion thereof contains an amino acid sequence of at least about 3 to about 5 amino acids, at least about 8 to about 10 amino acids, or at least about 15 to about 20 amino acids from a polypeptide encoded by the nucleic acid sequence. Also encompassed are polypeptide sequences which are immunologically identifiable with a polypeptide encoded by the sequence.

“Operably linked” as used herein refers to an arrangement of elements wherein the components so described are configured so as to perform their usual function. Thus, a given promoter that is operably linked to a coding sequence (e.g., a reporter expression cassette) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

A “vector” as used herein, is capable of transferring gene sequences to target cells. Typically, “vector construct”, “expression vector”, and “gene transfer vector”, mean any nucleic acid construct capable of directing the expression of a gene of interest and which can transfer gene sequences to target cells. Thus, the term includes cloning, and expression vehicles, as well as integrating vectors.

An “expression cassette” as used herein comprises any nucleic acid construct capable of directing the expression of a gene/coding sequence of interest. Such cassettes can be constructed into a “vector”, “vector construct”, “expression vector”, or “gene transfer vector”, in order to transfer the expression cassette into target cells. Thus, the term includes cloning and expression vehicles, as well as viral vectors.

A “tandem repeat” as used herein is a repetition of more than one nucleotide (in a nucleic acid) or more than one amino acid residue (in a protein), where the repetitions occur adjacent to each other in the sequence. Tandem repeats may be consecutive (i.e., with no other nucleotides or residues between the repeats), or the tandem repeats may be separated by one or more nucleotides or residues between the repeats.

The term “expression vector” as used herein refers to any suitable recombinant expression vector that can be used to transform or transfect a suitable host cell. The term “host cell”, as used herein, refers to a cell into which a recombinant expression vector has been introduced. The term “host cell” refers not only to the cell in which the expression vector is introduced (the “parent” cell), but also to the progeny of such a cell. Because modifications may occur in succeeding generations, for example, due to mutation or environmental influences, the progeny may not be identical to the parent cell but are still included within the scope of the term “host cell”.

The term “transformed” as used herein, means a heritable alteration in a cell resulting from the uptake of foreign DNA. Suitable methods for transformation of cells include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

An “immortalized cell” as used herein refers to a cell of a type that would not normally proliferate indefinitely, but having a mutation that allows it to evade cellular senescence so that it can continue undergoing cell division indefinitely. In embodiments, immortalized cell lines can be derived from tumor cell lines or can be derived from a cell line that is manipulated to allow the cells to proliferate indefinitely.

“Knock-in” as used herein refers to a transgenic cell or animal generated by a genetic engineering method that involves the insertion of a heterologous DNA sequence at a specific genomic location. In one aspect, the heterologous DNA sequence is inserted by homologous recombination. In one aspect, the heterologous DNA sequence is inserted using a CRISPR/Cas9 system. In one aspect, the heterologous DNA sequence is inserted into a “safe harbor locus.” A “safe harbor locus” as used herein is a site in the genome able to accommodate the integration of new genetic material so that the new genetic elements function predictably and do not cause alterations of the host genome posing a risk to the host cell or organism. “Knock-in” includes progeny that comprise the heterologous DNA sequence in at least one allele. In embodiments, by adding a reporter gene to this locus, it is possible to trace the lineage of the cell.

“Heterologous” as used herein refers to a nucleic acid that is not naturally occurring within a cell or animal, or a nucleic acid that is native to a cell or an animal, but has been altered or mutated.

“Transmembrane domain” (TM domain) as used herein refers to a generally hydrophobic region of a protein that crosses the plasma membrane of a cell. In embodiments, the TM domain links an extracellular portion of a construct to an intracellular portion. In embodiments, the TM domain links an extracellular affinity tag and an intracellular fluorescent reporter protein. The TM domain can include a transmembrane region of a protein, a fragment of a transmembrane of a protein, an artificial hydrophobic sequence, or a combination thereof. In embodiments, the transmembrane domain is a Type I transmembrane protein. In embodiments, the TM domain includes one or more α-helices. In embodiments, the TM domain includes one or more β-strands. In embodiments, the transmembrane domain includes an IgG transmembrane domain. In embodiments, the transmembrane domain includes a human IgG transmembrane domain. In embodiments, the transmembrane domain includes a mouse IgG transmembrane domain.

In embodiments, the transmembrane domain includes a mammalian transmembrane domain. In embodiments, the transmembrane domain includes the transmembrane domain of mouse proteins Tmem53, Lrtm1 or Nrg1. Although specific examples are provided herein, other transmembrane domains will be apparent to those of skill in the art and can be used in connection with the construct described herein. See, for example, Yu and Zhang (2013) “A simple method for predicting transmembrane proteins based on wavelet transform,” Int. J. Biol. Sci. 9(1):22-33.

### B. B Cell Development

In embodiments, the cells identified and/or isolated using the methods described herein are B cells. B cells develop from hematopoietic stem cells (HSCs) in the bone marrow where they undergo several phases of antigen-independent development, leading to the generation of immature B cells. Immature B cells express IgM on their surfaces (membrane IgM expression). Immature B cells migrate from the bone marrow into the spleen where they differentiate into mature naïve B cells (which express membrane IgM and IgD). Some of these mature naïve B cells differentiate into memory B cells-long-lived and quiescent cells that are capable of quickly activating upon re-exposure to the antigen, proliferating and differentiating into plasma cells to fight the new infection. When a naïve or memory B cell is activated by antigen, it proliferates and differentiates into an antibody-secreting cell.

Later, when cells are fully maturated to plasma cells, they express secreted Ig but lose Ig surface expression. About 99% of antibody expressing cells use Ig kappa as the light chain in the mouse and rat.

After HSCs are committed to the B cell lineage, B cell progenitors go through a series of differentiation events to become mature B cells.

### C. Homologous Recombination and Site Specific Nucleases

As described herein, the disclosure provides method of generating a genetically modified non-human mammal cells and organisms, where the methods involve introducing a nuclease into the non-human mammal cell, wherein the nuclease causes a single strand break or a double strand break at a location in the genome of the cell being modified. In embodiments, the repair of this single strand break or double strand break causes a nucleic acid sequence to be integrated into the genome of the cell being modified. In embodiments, this integration occurs via homologous recombination.

Homologous recombination (HR): Homologous recombination allows for the insertion of a target gene at a certain site within a genome of an organism (gene targeting). By creating DNA constructs that contain a template that matches the targeted genome sequence it is possible that the HR processes within the cell will insert the construct at the desired location. Using this method on embryonic stem cells led to the development of transgenic mice with targeted genes knocked out, i.e., removed from the genome or knocked in, i.e., added to the genome.

Methods of gene knock in using HR following a double strand break are described in the art, for example, as described in U.S. Pat. Nos. 5,474,896; 5,792,632; 5,866,361; 5,948,678; 5,948,678, 5,962,327; 6,395,959; 6,238,924; and 5,830,729, which are hereby incorporated by reference herein. Exemplary methodologies for homologous recombination are described in U.S. Pat. Nos. 6,689,610; 6,204,061; 5,631,153; 5,627,059; 5,487,992; and 5,464,764, each of which is incorporated by reference.

In embodiments, the single strand break or double strand break is introduced using a site specific nuclease. Such nucleases are known in the art and examples of such nucleases are provided herein.

Zinc finger nucleases: Zinc-finger nucleases have DNA binding domains that can precisely target a DNA sequence. Each zinc finger can recognize portions of a desired DNA sequence, and therefore can be modularly assembled to bind to a particular sequence. The binding domains guide the cutting of a restriction endonuclease that causes a double stranded break in the DNA.

Transcription activator-like effector nucleases (TALENs): Transcription activator-like effector nucleases (TALENs) also contain a DNA binding domain and a nuclease that can cleave DNA. The DNA binding region includes amino acid repeats that each recognize a single base pair of the desired targeted DNA sequence. The nuclease causes a double stranded break in the DNA.

CRISPR/Cas: Clustered regularly interspaced short palindromic repeats (CRISPR)/CRISPR-associated protein (Cas) is a method for genome editing that contains a guide RNA complexed with a Cas protein. The guide RNA can be engineered to match a desired DNA sequence through simple complementary base pairing, as opposed to the required assembly of constructs required by zinc-fingers or TALENs. The coupled Cas will cause a double stranded break in the DNA. In embodiments, the Cas protein includes Cas9. In embodiments, the Cas protein includes Cas9, Cas12 Cas12a, Cas13, Cas14 or CasΦ. In embodiments, the Cas protein includes Cas3, Cas8, Cas10, Cas11, Cas12, Cas12a, Cas13, Cas14 or CasΦ.

### D. Cre Recombinase

Cre (Cre recombinase) is one of the tyrosine site-specific recombinases (T-SSRs) including flipase (Flp) and D6 specific recombinase (Dre). It was discovered as a 38-kDa DNA recombinase produced from the cre (cyclization recombinase) gene of bacteriophage P1. It recognizes the specific DNA fragment sequences called loxP (locus of x-over, P1) site and mediates site-specific deletion of DNA sequences between two loxP sites. The loxP site is a 34 bp sequences that includes a two 13 bp inverted and palindromic repeats and 8 bp core sequences.

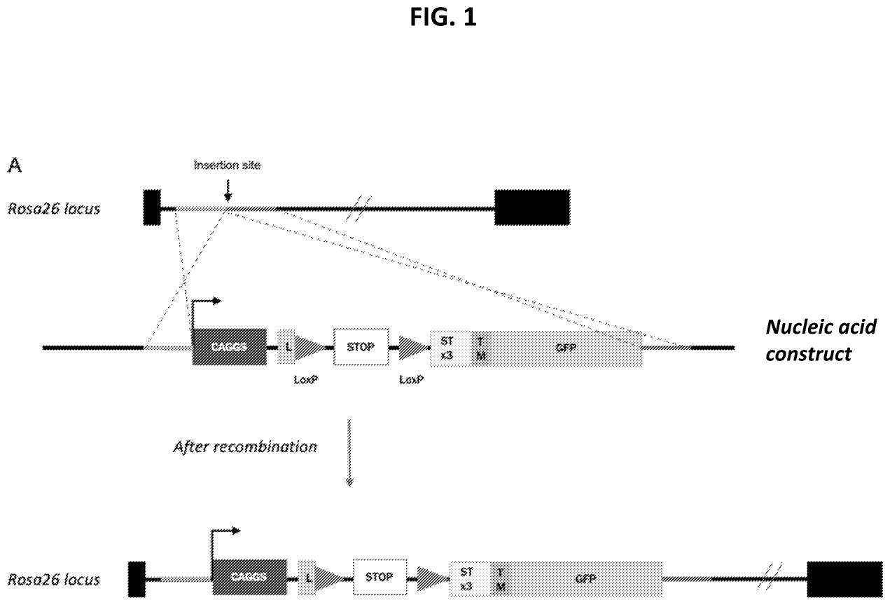

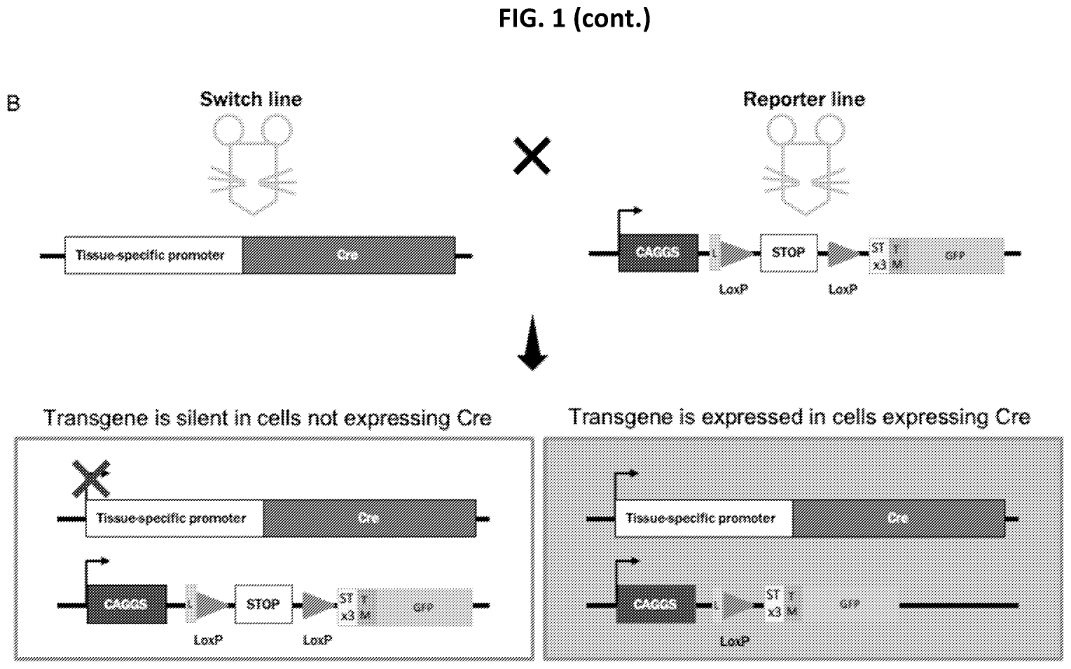

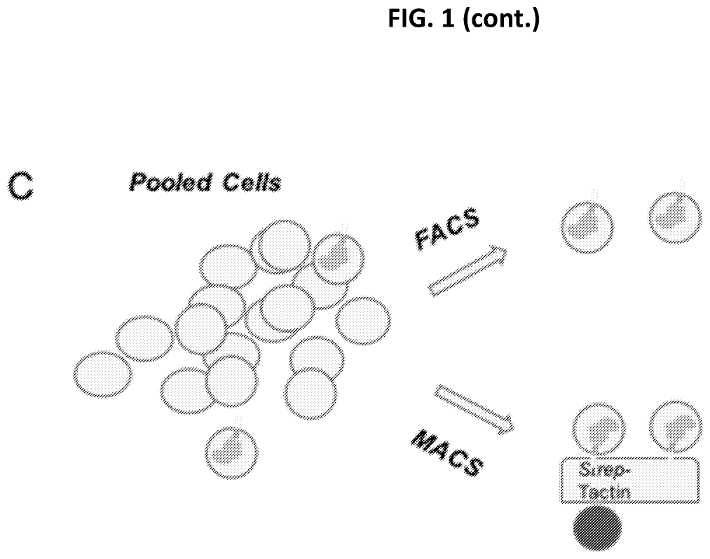

As described further herein, the proper insertion of a loxP-flanked “stop” sequence (transcriptional termination element) between the leader sequence and transgene coding reporter sequence blocks the expression of the gene. After conditional reporter line is bred with switch line, the cre recombinase removes the stop element in front of the reporter to switch on expression of the reporter within the nucleus of cre expressing cells. As a result, these cells are permanently labeled with affinity tag on the cell surface and the intracellular fluorescence marker. This process is shown schematically in FIG. 1B.

Thousands of mice lines have been developed where Cre is under control of a tissue specific promoter. Thus, Cre is only expressed in specific tissues in the mouse. As further described herein, by breeding to different switch lines, different cells of interest can be labeled with the reporter protein and isolated with large scale magnetic based methods or by flow based methods.

### E. Rodent Immunoglobulins

As in humans, there are five antibody isotypes (IgA, IgD, IgE, IgG, and IgM) in mice and rats. Each isotype has a different heavy chain. Isotypes may also be called classes. Naive B cells produce IgM and IgD. During B cell maturation, through isotypic switching, a mature B cell will produce one of IgG, or IgA, or IgE isotypes and subclasses. Different isotypes have different half-lives in vivo, ranging from 12 hours to 8 days.

Heavy chains for IgA, IgD, and IgG have a constant region with three immunoglobulin (Ig) domains. Other types of heavy chains may have a different number of immunoglobulin domains. Heavy chains for IgE and IgM have a constant region with four immunoglobulin domains. Each of the heavy chain of the isotypes above has a membrane bound version and a secreted version at the C-terminal region via alternative splicing event occurred during the transcription. The membrane bound version mRNA includes 2 additional exons at the C-terminal ends; therefore, the protein of the membrane bound version heavy chain is longer with a transmembrane domain and a cytosolic C-terminal tail. Heavy chains from all isotypes have a variable region with a single immunoglobulin domain.

Each light chain (either kappa or lambda) has one constant immunoglobulin domain and one variable immunoglobulin domain. In rat and mouse, the light chain usage between Kappa to lambda is roughly 99 to 1 meaning approximately 99% of antibody expressing cells express the Kappa light chain. The murine immunoglobulin kappa (kappa) light-chain multigene family includes the constant region locus (C kappa), 4 joining-region genes, and approximately 95 kappa-variable (V kappa) region families.

### F. Transgenic Animals

A “transgenic animal” is a non-human animal, usually a mammal, having an exogenous nucleic acid sequence present as an extrachromosomal element in a portion of its cells or stably integrated into its germ line DNA (i.e., in the genomic sequence of most or all of its cells). In embodiments herein, the transgenic animal comprises exogenous nucleic acid introduced into the germ line of such transgenic animals by genetic manipulation of, for example, embryos or embryonic stem cells of the host animal according to methods well known in the art. In embodiments herein, the transgenic animals comprise more than the nucleic acid reporter constructs described herein. In embodiments, the transgenic animals comprise one or more additional nucleic acids encoding a product to be produced by the transgenic animal, for example, a protein, such as an enzyme or immunoglobulin, or a nucleic acid, such as a DNA or RNA. In specific aspects, methods herein provide for the creation of transgenic animals comprising the introduced partially human immunoglobulin region along with a nucleic acid encoding a reporter construct as described herein.

In embodiments, the transgenic animals are rodents, e.g., mice or rats. In embodiments, the transgenic rodents comprise endogenous mouse immunoglobulin regions with human immunoglobulin sequences to create partially- or fully-human antibodies for drug discovery purposes. Examples of such mice include those described in, for example, U.S. Pat. Nos. 7,145,056; 7,064,244; 7,041,871; 6,673,986; 6,596,541; 6,570,061; 6,162,963; 6,130,364; 6,091,001; 6,023,010; 5,593,598; 5,877,397; 5,874,299; 5,814,318; 5,789,650; 5,661,016; 5,612,205; and 5,591,669, which are hereby incorporated by reference. In embodiments, the transgenic rodents are transgenic mice whose genome comprises an entire endogenous mouse immunoglobulin locus variable region which has been deleted and replaced with an engineered immunoglobulin locus variable region. Examples of such mice include those described in, for example, U.S. Pat. No. 10,881,084 and U.S. Pat. Pub. 2020/0190218 which are hereby incorporated by reference. In embodiments, the transgenic mice are engineered to express human or partially-human antibodies. In other embodiments, the transgenic mice are engineered to express dog, horse or cow antibodies. Examples of such mice include those described in, for example, U.S. Pat. No. 10,793,829, U.S. Pat. Pub Nos. 2020/0308307 and 2021/0000087 and Intl. Pat. Pub. No. WO2021/003152, which are hereby incorporated by reference.

### G. Cell Sorting Methods

Fluorescence-activated cell sorting (FACS) is a specialized type of flow cytometry. This method is capable of sorting a heterogeneous mixture of cells into two or more containers, one cell at a time, based upon the specific light scattering and fluorescent characteristics of each cell. FACS is performed using cell sorting instruments designed for the technique. FACS provides fast, objective and quantitative recording of fluorescent signals from individual cells as well as physical separation of cells of particular interest.

In embodiments, FACS is generally performed as follows. A suspension of the cells to be sorted is entrained in the center of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell per droplet. Just before the stream breaks into droplets, the flow passes through a fluorescence measuring station where the fluorescent character of interest of each cell is measured. An electrical charging ring is placed just at the point where the stream breaks into droplets. A charge is placed on the ring based on the immediately prior fluorescence intensity measurement, and the opposite charge is trapped on the droplet as it breaks from the stream. The charged droplets then fall through an electrostatic deflection system that diverts droplets into containers based upon their charge. In some systems, the charge is applied directly to the stream, and the droplet breaking off retains charge of the same sign as the stream. The stream is then returned to neutral after the droplet breaks off and the next droplet is measured and sorted.

Magnetic-activated cell sorting (MACS; Miltenyi Biotech) is a method for separation of cells by markers on the surface of the cells. In embodiments, the MACS system uses superparamagnetic nanoparticles and columns. The superparamagnetic nanoparticles are of the order of 100 nm. The nanoparticles tag the targeted cells in order to capture them inside the column. The column is placed between permanent magnets so that when the magnetic particle-cell complex passes through it, the tagged cells can be captured. The magnetic nanoparticles are coated with agents that bind a specific marker on their surface. Cells expressing the marker attach to the magnetic nanoparticles. After incubating the beads and cells, the solution is transferred to a column in a strong magnetic field. The cells attached to the nanoparticles (expressing the marker) stay on the column, while other cells (not expressing the marker) flow through.

In embodiments, the cells are sorted using the affinity tag expressed on the surface of the cells. Affinity tags for this purpose are described herein. In these embodiments, the cells can be sorted using an affinity purification column or resin that binds to the affinity tag using methods known in the art. As a non-limiting example, when the cells express a StrepII tag on their surface, the cells can be captured, and therefore sorted, using a resin that binds the StrepII tag, e.g. Strep-Tactin® Sepharose® (IBA Lifesciences).

### H. Conditional Reporter Nucleic Acid Constructs

In embodiments, provided herein is a nucleic acid construct comprising a leader sequence, a LoxP-Stop-LoxP cassette, and a transmembrane reporter cassette encoding an affinity tag, a transmembrane (TM) domain and a fluorescent reporter protein. Embodiments of this nucleic acid construct may be referred to herein as a “conditional reporter nucleic acid construct.”

In embodiments of the construct, the leader sequence is upstream of the LoxP-Stop-LoxP cassette. In embodiments, the leader sequence is downstream of the LoxP-Stop-LoxP cassette.

In embodiments, the LoxP-Stop-LoxP cassette comprises a stop element, e.g., any type of sequence that causes translation or transcription to terminate. In embodiments, the stop element comprises one or more SV40 polyadenylation sequences.

In embodiments, the LoxP-Stop-LoxP cassette comprises two LoxP sites flanking a sequence that causes termination of transcription. In embodiments, the LoxP-Stop-LoxP cassette comprises a LoxP flanked polyadenylation sequence that causes termination of transcription. In embodiments, the polyadenylation signal is a SV40, hGH, BGH or rbGlob polyadenylation signal. In embodiments, the LoxP-Stop-LoxP cassette comprises a LoxP-flanked triple repeat of the polyadenylation sequence. In embodiments, the LoxP-Stop-LoxP cassette comprises a LoxP-flanked double repeat of the polyadenylation sequence. In embodiments, the LoxP-Stop-LoxP cassette comprises a LoxP-flanked single polyadenylation sequence.

In embodiments, the LoxP-Stop-LoxP cassette comprises a LoxP-flanked triple repeat of the SV40 polyadenylation sequence. In embodiments, the LoxP-Stop-LoxP cassette comprises a LoxP-flanked double repeat of the SV40 polyadenylation sequence. In embodiments, the LoxP-Stop-LoxP cassette comprises a LoxP-flanked single SV40 polyadenylation sequence.

In embodiments, the stop element comprises one or more stop codons that cause termination of translation. In embodiments, the LoxP-Stop-LoxP cassette comprises a LoxP-flanked stop codon. In embodiments, the stop codon is TAG, TAA or TGA.

In embodiments, the nucleic acid construct comprises single stranded DNA, double stranded DNA, a plasmid, or a viral vector. In embodiments, the nucleic acid construct is linear DNA. In embodiments, the nucleic acid construct is circular DNA.

In embodiments, the nucleic acid construct further comprises a first homology arm and a second homology arm that are homologous to a first target sequence and a second target sequence in the genome of a non-human mammal. The homologous regions allow for integration of the nucleic acid construct into the genome of the non-human mammal using methods described herein and known in the art. In embodiments, the nucleic acid construct further comprises a first homology arm and a second homology arm that are homologous to a first target sequence and a second target sequence, respectively, within a safe harbor locus in a non-human mammal.

In embodiments, the first homology and second homology arms, each independently, comprise from about 15 nucleotides to about 12000 nucleotides. In embodiments, the first homology and second homology arms, each independently, comprise from about 30 nucleotides to about 11000 nucleotides. In embodiments, the first homology and second homology arms, each independently, comprise from about 50 nucleotides to about 10000 nucleotides. In embodiments, the first homology and second homology arms, each independently, comprise from about 100 nucleotides to about 7500 nucleotides. In embodiments, the first homology and second homology arms, each independently, comprise from about 200 nucleotides to about 5000 nucleotides. In embodiments, the first homology and second homology arms, each independently, comprise from about 300 nucleotides to about 2500 nucleotides.

In embodiments, the safe harbor locus is any site in the genome able to accommodate the integration of new genetic material so that the new genetic elements function predictably and do not cause alterations of the host genome posing a risk to the host cell or organism. In embodiments, the safe harbor locus is a mouse safe harbor locus. In embodiments, the safe harbor locus is a rat safe harbor locus. In embodiments, the safe harbor locus comprises a Rosa26 locus on chromosome 6 in a genome of a mouse. In embodiments, the safe harbor locus comprises a Hipp11 locus on chromosome 11 in a genome of a mouse.

In embodiments, the nucleic acid construct is expressed using an endogenous promoter. In embodiments, the nucleic acid construct is expressed using an endogenous promoter located in the safe harbor locus.

In embodiments, the nucleic acid construct further comprises a promoter. In embodiments, the promoter is a mammalian constitutive promoter. In embodiments, the promoter is a human promoter. In embodiments, the promoter is a mouse promoter. In embodiments, the promoter is a rat promoter. In embodiments, the promoter is a viral promoter. In embodiments, the promoter comprises a CAG promoter. In embodiments, the promoter comprises a CAG, CMV, EF1a, SV40, PGK1, Ubc or human beta actin promoter.

In embodiments, the leader sequence comprises a secretory signal peptide. In embodiments, the secretory signal peptide is an IL-2 leader sequence. In embodiments, the secretory signal peptide is a human OSM, VSV-G mouse Ig Kappa, Human IgG2 H, BM40, Secrecon, human IgKVIII, CD33, tPA, human chymotrypsinogen, human trypsinogen-2, human IL-12 or a human serum albumin signal peptide. In embodiments, the secretory signal peptide comprises the IL-2 leader sequence MYRMQLLSCIALSLALVINS (SEQ ID NO: 2). Those skilled in the art will understand that signal peptides can be predicted using algorithms known in the art, e.g. the SignalP-5.0 Server at www.cbs.dtu.dk/services/SignalP/and the SecretomeP 2.0 Server at www.cbs.dtu.dk/services/SecretomeP/.

The affinity tag of the nucleic acid construct can be used for later isolation or purification of a protein expressed by the construct. In embodiments, the affinity tag comprises a StrepII, hexahistadine, FLAG, HA, Myc, VA, GST, beta-GAL, MBP or VSV-G tag. In embodiments, the affinity tag comprises from about 1 to about 18 tandem repeats of a tag. In embodiments, the affinity tag comprises from about 2 to about 15 tandem repeats of a tag. In embodiments, the affinity tag comprises from about 3 to about 10 tandem repeats of a tag. In embodiments, the affinity tag comprises 3 tandem repeats of a tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a tag.

In embodiments, the affinity tag comprises a StrepII-tag. In embodiments, the affinity tag comprises tandem repeats of a StrepII-tag. In embodiments, the affinity tag comprises from about 1 to about 18 tandem repeats of a StrepII-tag. In embodiments, the affinity tag comprises from about 2 to about 15 tandem repeats of a StrepII-tag. In embodiments, the affinity tag comprises from about 3 to about 10 tandem repeats of a StrepII-tag. In embodiments, the affinity tag comprises 3 tandem repeats of a StrepII-tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a StrepII-tag. In embodiments, the tandem repeats have a tag linker in between repeats. In embodiments, the linker is a dipeptide or a tripeptide. In embodiments, the tag linker is the dipeptide Ser-Ala.

In embodiments, the StrepII-tag comprises an eight amino acid peptide sequence of WSHPQFEK (SEQ ID NO: 1). In embodiments, the affinity tag comprises WSHPQFEKSAWSHPQFEKSAWSHPQFEK (SEQ ID NO: 3).

The transmembrane domain encoded by the transmembrane reporter cassette allows the affinity tag to be presented on the surface of cells expressing the nucleic acid construct. In embodiments, the transmembrane domain comprises a hydrophobic α-helix. In embodiments, the transmembrane domain includes an IgG transmembrane domain. In embodiments, the transmembrane domain includes a human IgG1 transmembrane domain. In embodiments, the transmembrane domain includes a mouse IgG transmembrane domain. In embodiments, the transmembrane domain includes a mouse IgG1, IgG2a, IgG2b or IgG2c transmembrane domain. In embodiments, the transmembrane domain includes the transmembrane domain of the mouse proteins Tmem53, Lrtm1 or Nrg1.

The fluorescent reporter protein allows detection of cells expressing the nucleic acid construct. In embodiments, the fluorescent reporter protein comprises a green fluorescent protein, a yellow fluorescent protein, a cyan fluorescent protein, a red fluorescent protein, a blue fluorescent protein, a red fluorescent protein or an orange fluorescent protein. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP). In embodiments, the fluorescent reporter protein comprises red fluorescent protein (RFP). In embodiments, the red fluorescent protein is monomeric cherry (mCherry) or tandem dimer Tomato (tdTomato).

In embodiments, the nucleic acid construct is the nucleic acid construct shown schematically in FIG. 1A, where CAGGS represents a CAG promoter, L represents a leader, the LoxP-Stop-LoxP cassette arranged as shown, STX3 represents a three tandem repeats of the Strep-II tag, TM represents a transmembrane domain and GFP represents a green fluorescent protein reporter.

### I. Methods of Generating Conditional Reporter Modified Cells and Organisms

In embodiments, provided herein is a method of generating a genetically modified non-human mammal cell, the method comprising:
(a) introducing a conditional reporter nucleic acid construct described herein into the non-human mammal cell; and
(b) introducing a nuclease into the non-human mammal cell, wherein the nuclease causes a single strand break or a double strand break at a safe harbor locus in a genome of the non-human mammal cell, wherein the nucleic acid construct is integrated into the genome of the non-human mammal cell at the safe harbor locus by homologous recombination.

In embodiments, the nuclease causes a double strand break. In embodiments, the nuclease causes a single strand break (e.g., a nick).

The nuclease may be introduced into the cell using methods known in the art. In embodiments, introducing the nuclease comprises introducing an expression construct encoding the nuclease. In embodiments, the introducing of the expression construct is via injection or electroporation. In embodiments, introducing the nuclease comprises introducing a plasmid encoding the nuclease. In embodiments, introducing the nuclease comprises introducing a viral vector encoding the nuclease. In embodiments, introducing the nuclease comprises introducing a mRNA encoding the nuclease. In embodiments, the mRNA comprises one or more modified bases. In embodiments, the mRNA is encapsulated in a lipid nanoparticle. In embodiments, the introducing of the plasmid, viral vector or mRNA is via injection or electroporation. In embodiments, introducing the nuclease comprises introducing the nuclease protein directly into the cell. In embodiments, the introducing of the nuclease protein directly into the cell is via injection or electroporation.

In embodiments, the nuclease is a nuclease as described herein. In embodiments, the nuclease comprises a Zinc Finger nuclease (ZFN), a transcription activator-Like Effector Nuclease (TALEN), a Meganuclease, or a Clustered Regularly Interspaced Short Palindromic Repeats (CRISPR)-associated (Cas) protein and a guide RNA (gRNA). In embodiments, the gRNA comprises a CRISPR RNA (crRNA) that targets a recognition site and a trans-activating CRISPR RNA (tracrRNA). In embodiments, the CRISPR-Cas protein comprises Cas9. In embodiments, the Cas protein includes Cas9, Cas12 Cas12a, Cas13, Cas14 or CasΦ. In embodiments, the CRISPR-Cas protein comprises Cas3, Cas8, Cas10, Cas11, Cas12, Cas12a, Cas13, Cas14 or CasΦ.

In embodiments, the non-human mammal cell is from a mammal used in scientific research. In embodiments, the non-human mammal cell is a rodent cell. In embodiments, the rodent cell is a rat cell or a mouse cell.

In embodiments, the safe harbor locus is any locus able to accommodate the integration of new genetic material so that the new genetic elements function predictably and do not cause alterations of the host genome posing a risk to the host cell or organism. In embodiments, the safe harbor locus is a mouse safe harbor locus. In embodiments, the safe harbor locus is a rat safe harbor locus. In embodiments, the safe harbor locus comprises a Rosa26 locus on chromosome 6. In embodiments, the safe harbor locus comprises a Hipp11 locus on chromosome 11 in a genome of a mouse.

In embodiments, the non-human mammal cell is a pluripotent cell. In embodiments, the pluripotent cell is a non-human zygote. In embodiments, the pluripotent cell is a mouse zygote. In embodiments, the pluripotent cell is a rat zygote. In embodiments, the pluripotent cell is a non-human embryonic stem (ES) cell. In embodiments, the pluripotent cell is a mouse embryonic stem (ES) cell or a rat embryonic stem (ES) cell.

In embodiments, the zygote is injected with a nucleic acid construct described herein. In embodiments, the nucleic acid construct is injected into a pronucleus of the zygote. In embodiments, the microinjected zygote is implanted in the oviduct of a pseudo-pregnant female rodent. In embodiments, the pseudo-pregnant female rodent is a mouse. In embodiments, the pseudo-pregnant female rodent is a rat. In embodiments, the implanted zygote develops into a fetus and is birthed to provide a genetically modified non-human mammal. In embodiments, the genetically modified non-human mammal is a mouse. In embodiments, the genetically modified non-human mammal is a rat.

In embodiments, the method of generating a genetically modified non-human mammal cell further comprises isolating the genetically modified non-human mammal cell in which the nucleic acid construct is integrated at the safe harbor locus.

In embodiments, also provided herein is a genetically modified a non-human mammal cell generated by the above method of generating a genetically modified non-human mammal cell. In embodiments, the non-human mammal is a rodent. In embodiments, the non-human mammal is a mouse or rat.

In embodiments of the method of generating a genetically modified non-human mammal cell, the method further comprises steps for generating a transgenic non-human mammal. In embodiments, the method further comprises injecting the isolated cell into a blastocyst and generating a transgenic non-human mammal comprising the nucleic acid construct integrated into the safe harbor locus.

In embodiments, the disclosure provides a genetically modified non-human transgenic mammal generated by this method. In embodiments, the transgenic mammal is a rodent. In embodiments, the rodent is a rat or a mouse.

In embodiments of the method for generating a transgenic non-human mammal, the method further comprises breeding the transgenic non-human mammal comprising the nucleic acid construct integrated into the safe harbor locus with a transgenic non-human mammal that expresses Cre recombinase to obtain a non-human mammal with cells that express a fusion protein comprising an affinity tag, a transmembrane domain and a fluorescent reporter protein.

In embodiments of the method, the transgenic non-human mammal comprising the nucleic acid construct integrated into the safe harbor locus is a mouse comprising the nucleic acid construct integrated into a Rosa26 locus and the transgenic non-human mammal that expresses Cre recombinase is a mouse.

In embodiments of the method, the transgenic non-human mammal comprising the nucleic acid construct integrated into the safe harbor locus is a mouse comprising the nucleic acid construct integrated into a Hipp11 locus and the transgenic non-human mammal that expresses Cre recombinase is a mouse.

In embodiments, a transgenic non-human mammal that expresses Cre recombinase expresses Cre-recombinase under control of a tissue specific promoter. In embodiments, a transgenic non-human mammal that expresses Cre recombinase expresses Cre-recombinase under control of a promoter that is active only at a certain time during cell development.

In embodiments, the transgenic non-human mammal is a Cre switch line mouse, for example, a Cre switch line found in the Mouse Genome Informatics database: www.informatics.jax.org/home/recombinase. In embodiments, the Cre switch line mouse is a Blimp1-CreER12 mouse line. As a non-limiting example, this Blimp1-CreERT2 can be used to breed with the genetically modified non-human transgenic mammal described above to label plasmablasts and plasma cells, which express the Blimp1 transcription factor. In embodiments, the Cre switch line mouse is a JchaincreERT2 mouse line. In embodiments, the JchaincreERT2 mouse can be bred with the genetically modified non-human transgenic mammals described herein to more specifically label plasma cells including all immunoglobulin isotypes. In embodiments, the Cre switch line mouse is an Xbp1 mouse line. In embodiments, the Cre switch line mouse is an Irf4 mouse line.

In embodiments, Cre expression in the transgenic mouse is tissue specific. In embodiments, Cre expression in the transgenic mouse is specific to the state of cell development.

In embodiments, the method for generating a transgenic non-human mammal comprising breeding the transgenic non-human mammal comprising the nucleic acid construct integrated into the safe harbor locus with a transgenic non-human mammal that expresses Cre recombinase to obtain a non-human mammal with cells that express a fusion protein comprising an affinity tag, a transmembrane domain and a fluorescent reporter protein is performed as represented by the schematic in FIG. 1B. The transgenic non-human mammal generated using this method functions as shown in FIG. 1B. The transgene is silent in cells where the tissue specific promoter is not active, as the stop sequence remains in the construct. When the tissue specific promoter is expressed, the expressed Cre excises the stop sequence, causing the transgene to be expressed.

In embodiments, provided herein is a genetically modified non-human mammal with cells that express a fusion protein comprising an affinity tag, a transmembrane domain and a fluorescent reporter protein generated by the methods described above.

### J. Conditional Reporter Modified Cells

In embodiments, provided herein is a genetically modified non-human mammal cell comprising a genome comprising a conditional reporter nucleic acid construct described herein integrated into a safe harbor locus. In embodiments of the cell, the safe harbor locus comprises a Rosa26 locus on chromosome 26 in a genome of a mouse. In embodiments of the cell, the safe harbor locus comprises a Hipp11 locus on chromosome 11 in a genome of a mouse.

In embodiments, the cell is a hybridoma. In embodiments, the cell is a stem cell. In embodiments, the stem cell is an embryonic stem cell. In embodiments, the stem cell is an adult stem cell. In embodiments, the stem cell is an induced pluripotent stem cell. In embodiments, the stem cell is a perinatal stem cell. In embodiments, the cell is an immortalized cell.

In embodiments, the genetically modified non-human mammal cell expresses a fusion protein comprising an affinity tag, a transmembrane domain and a fluorescent reporter protein. In embodiments, the affinity tag is expressed on a cell surface of the non-human mammal cell. Examples of affinity tags that can be expressed on the cell surface of the non-human mammal cell are described herein.

In embodiments, the affinity tag comprises a StrepII, hexahistadine, FLAG, HA, Myc, VA, GST, beta-GAL, MBP or VSV-G tag. In embodiments, the affinity tag comprises from about 1 to about 18 tandem repeats of a tag. In embodiments, the affinity tag comprises from about 2 to about 15 tandem repeats of a tag. In embodiments, the affinity tag comprises from about 3 to about 10 tandem repeats of a tag. In embodiments, the affinity tag comprises 3 tandem repeats of a tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a tag.

In embodiments, the affinity tag comprises a StrepII-tag. In embodiments, the affinity tag comprises tandem repeats of a StrepII-tag. In embodiments, the affinity tag comprises from about 1 to about 18 tandem repeats of a StrepII-tag. In embodiments, the affinity tag comprises from about 2 to about 15 tandem repeats of a StrepII-tag. In embodiments, the affinity tag comprises from about 3 to about 10 tandem repeats of a StrepII-tag. In embodiments, the affinity tag comprises 3 tandem repeats of a StrepII-tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a StrepII-tag. In embodiments, the tandem repeats have a tag linker in between repeats. In embodiments, the linker is a dipeptide or a tripeptide. In embodiments, the tag linker is the dipeptide Ser-Ala.

In embodiments, the StrepII-tag comprises an eight amino acid peptide sequence of WSHPQFEK (SEQ ID NO: 1). In embodiments, the affinity tag comprises WSHPQFEKSAWSHPQFEKSAWSHPQFEK (SEQ ID NO: 3).

In embodiments, the fluorescent reporter protein is exposed on a cytosolic surface of the non-human mammal cell.

In embodiments, the fluorescent reporter protein comprises a green fluorescent protein, a yellow fluorescent protein, a cyan fluorescent protein, a red fluorescent protein, a blue fluorescent protein, a red fluorescent protein or an orange fluorescent protein. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP). In embodiments, the fluorescent reporter protein comprises red fluorescent protein (RFP). In embodiments, the red fluorescent protein is monomeric cherry (mCherry) or tandem dimer Tomato (tdTomato).

### K. Methods for Isolating Conditional Reporter Modified Cells

In embodiments, provided herein is a method for isolating cells obtained from a genetically modified non-human mammal, the method comprising:
(a) obtaining cells from a genetically modified conditional reporter non-human mammal described herein;
(b) screening the cells obtained from the genetically modified non-human mammal for expression of a fusion protein comprising an affinity tag, a transmembrane domain and a fluorescent reporter protein; and
(c) isolating cells expressing the fusion protein.

In embodiments of the method for isolating cells, the cells are screened by fluorescent activated cell sorting (FACS) or magnetic activated cell sorting (MACS). In embodiments, of the method for isolating cells, the cells are screened by fluorescent activated cell sorting (FACS). In embodiments, of the method for isolating cells, the cells are screened by magnetic activated cell sorting (MACS). The techniques of both FACS and MACS are known in the art and are described elsewhere herein. In embodiments, separation of cells using either FACS or MACS is shown schematically in FIG. 1C. In embodiments of the methods of isolating cells, the cells are separated using the affinity tag expressed on the surfaces of the cells, as described herein. In embodiments where the cells are separated using the affinity tag, the cells are separated using an affinity column or an affinity resin that binds the affinity tag using methods known in the art.

In embodiments, the affinity tag is expressed on a cell surface of the genetically modified non-human mammal cell.

In embodiments, the affinity tag comprises a StrepII, hexahistadine, FLAG, HA, Myc, VA, GST, beta-GAL, MBP or VSV-G tag. In embodiments, the affinity tag comprises from about 1 to about 18 tandem repeats of a tag. In embodiments, the affinity tag comprises from about 2 to about 15 tandem repeats of a tag. In embodiments, the affinity tag comprises from about 3 to about 10 tandem repeats of a tag. In embodiments, the affinity tag comprises 3 tandem repeats of a tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a tag.

In embodiments, the affinity tag comprises a StrepII-tag. In embodiments, the affinity tag comprises tandem repeats of a StrepII-tag. In embodiments, the affinity tag comprises from about 1 to about 18 tandem repeats of a StrepII-tag. In embodiments, the affinity tag comprises from about 2 to about 15 tandem repeats of a StrepII-tag. In embodiments, the affinity tag comprises from about 3 to about 10 tandem repeats of a StrepII-tag. In embodiments, the affinity tag comprises 3 tandem repeats of a StrepII-tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a StrepII-tag. In embodiments, the tandem repeats have a tag linker in between repeats. In embodiments, the linker is a dipeptide or a tripeptide. In embodiments, the tag linker is the dipeptide Ser-Ala.

In embodiments, the StrepII-tag comprises an eight amino acid peptide sequence of WSHPQFEK (SEQ ID NO: 1). In embodiments, the affinity tag comprises WSHPQFEKSAWSHPQFEKSAWSHPQFEK (SEQ ID NO: 3).

In embodiments, the fluorescent reporter protein is exposed on a cytosolic surface of the non-human mammal cell.

In embodiments, the fluorescent reporter protein comprises a green fluorescent protein, a yellow fluorescent protein, a cyan fluorescent protein, a red fluorescent protein, a blue fluorescent protein, a red fluorescent protein or an orange fluorescent protein. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP). In embodiments, the fluorescent reporter protein comprises red fluorescent protein (RFP). In embodiments, the red fluorescent protein is monomeric cherry (mCherry) or tandem dimer Tomato (tdTomato).

### L. Immunoglobulin Reporter Nucleic Acid Constructs

In embodiments, provided herein is a nucleic acid construct comprising a linker, a leader sequence, and a transmembrane reporter cassette encoding an affinity tag, a transmembrane domain and a fluorescent reporter. Embodiments of this nucleic acid construct may be referred to herein as an “immunoglobulin reporter nucleic acid construct.”

In embodiments, the nucleic acid construct comprises single stranded DNA, double stranded DNA, a plasmid, or a viral vector. In embodiments, the nucleic acid construct is linear DNA. In embodiments, the nucleic acid construct is circular DNA.

In embodiments, the nucleic acid construct further comprises a first homology arm and a second homology arm that are homologous to a first target sequence and a second target sequence in the genome of a non-human mammal. The homologous regions allow for integration of the nucleic acid construct into the genome of the non-human mammal using methods described herein and known in the art. In embodiments, the nucleic acid construct further comprises a first homology arm and a second homology arm that are homologous to a first target sequence and a second target sequence, respectively, within an immunoglobulin locus in a non-human mammal, e.g. an immunoglobulin variable domain locus or an immunoglobulin constant domain locus or in between. In embodiments, the nucleic acid construct further comprises a first homology arm and a second homology arm that are homologous to a first target sequence and a second target sequence, respectively, within an immunoglobulin constant domain locus in a non-human mammal.

In embodiments, the first homology arm and a second homology arm that are homologous to a first target sequence and a second target sequence, respectively, wherein the first and second target sequences flank an immunoglobulin constant domain locus. In embodiments, the immunoglobulin constant domain locus is an immunoglobulin light chain constant domain locus. In embodiments, the immunoglobulin light chain constant domain locus is a kappa light chain constant domain locus. In embodiments, the immunoglobulin light chain constant domain locus is a lambda light chain constant domain locus. In embodiments, the immunoglobulin constant domain locus is an immunoglobulin heavy chain constant domain locus. In embodiments, the immunoglobulin heavy chain constant domain locus is a gamma, delta, alpha, mu or epsilon immunoglobulin constant domain locus.

In embodiments, the first target sequence is upstream of an immunoglobulin constant domain locus and the second target sequence is downstream of a stop codon of the immunoglobulin constant domain locus. In embodiments, the immunoglobulin constant domain locus is an immunoglobulin light chain constant domain locus. In embodiments, the immunoglobulin light chain constant domain locus is an immunoglobulin kappa constant domain locus. In embodiments, the immunoglobulin light chain constant domain locus is an immunoglobulin lambda constant domain locus.

In embodiments, the immunoglobulin constant domain locus is an immunoglobulin heavy chain constant domain locus. In embodiments, the immunoglobulin heavy chain constant domain locus is a gamma, delta, alpha, mu or epsilon immunoglobulin constant domain locus.

In embodiments, the first homology and second homology arms, each independently, comprise from about 15 nucleotides to about 12000 nucleotides. In embodiments, the first homology and second homology arms, each independently, comprise from about 30 nucleotides to about 11000 nucleotides. In embodiments, the first homology and second homology arms, each independently, comprise from about 50 nucleotides to about 10000 nucleotides. In embodiments, the first homology and second homology arms, each independently, comprise from about 100 nucleotides to about 7500 nucleotides. In embodiments, the first homology and second homology arms, each independently, comprise from about 200 nucleotides to about 5000 nucleotides. In embodiments, the first homology and second homology arms, each independently, comprise from about 300 nucleotides to about 2500 nucleotides.

In some embodiments, the linker comprises a stop codon and an Internal Ribosomal Entry Site (IRES). In embodiments, wherein the linker comprises a protease recognition site and a self-cleaving peptide. In embodiments, the linker comprises a leaky stop codon (LSC) with a peptide linker, a protease recognition site, and a self-cleaving peptide.

Embodiments of protease recognition sites are described herein. In embodiments, the protease recognition site comprises a Furin protease recognition site. In embodiments, the Furin protease recognition site comprises a nucleic acid sequence encoding the peptide of Arg-X-Arg-Arg. In embodiments, X is a hydrophobic amino acid. In embodiments, X is a hydrophilic amino acid. In embodiments, X is lysine. In embodiments, canonically, the Furin protease recognition site comprises a nucleic acid sequence encoding the peptide X-Arg-X-Lys-Arg-X or X-Arg-X-Arg-Arg-X. In embodiments, X is a hydrophobic amino acid. In embodiments, the hydrophobic amino acid comprises, Gly, Ala, Ile, Leu, Met, Val, Phe, Trp or Tyr. In embodiments, X is a hydrophilic amino acid. In embodiments, the hydrophilic amino acid is lysine. In embodiments, the Furin protease recognition site comprises a nucleic acid sequence encoding the peptide Arg-Lys-Arg-Arg. In embodiments, the Furin protease recognition site comprises a nucleic acid sequence encoding the peptide Arg-Arg-Arg-Arg. In embodiments, the Furin protease recognition site comprises a nucleic acid sequence encoding the peptide Arg-Arg-Lys-Arg. In embodiments, the Furin protease recognition site comprises a nucleic acid sequence encoding the peptide Arg-Lys-Lys-Arg. In embodiments, a Lys residue just prior to the Furin protease site is deleted. In embodiments, the Furin protease recognition site is a Furin protease recognition site as described in Fang et al., Molecular Therapy 15 (6): 1153-1159 (2007), which is hereby incorporated by reference herein.

In embodiments, the protease is an endoprotease. In embodiments, the protease is a mammalian endoprotease. In embodiment, the protease is an endoprotease endogenously expressed in a cell comprising the nucleic acid construct. In embodiments, the protease recognition site comprises a trypsin, chymotrypsin, elastase, thermolysin, pepsin, glutamyl endopeptidase or neprilysin recognition site.

Embodiments of self-cleaving peptides are described herein. In embodiments, the self-cleaving peptide comprises a 2A self-cleaving peptide. In embodiments, the self-cleaving peptide comprises a T2A (EGRGSLLTCGDVEENPGP; SEQ ID NO: 4), P2A (ATNFSLLKQAGDVEENPGP; SEQ ID NO: 5), E2A (QCTNYALLKLAGDVESNPGP; SEQ ID NO: 6) or an F2A (VKQTLNFDLLKLAGDVESNPGP; SEQ ID NO:7) self-cleaving peptide.

Embodiments of leaky stop codons are described herein. In embodiments, the sequence encoding the leaky stop codon comprises TGACTAG. In embodiments, the sequence encoding the leaky stop codon comprises TGACGG. In embodiments, the sequence encoding the leaky stop codon comprises TAGCAATTA. In embodiments, the sequence encoding the leaky stop codon comprises TAGCAATCA. In embodiments, the sequence encoding the leaky stop codon comprises TGACTA.

In embodiments where the linker comprises a leaky stop codon, the leaky stop codon allows for some read through of the codon, causing the transmembrane reporter cassette to be expressed. In embodiments, read through transcription of the leaky codon occurs about 5% of the time. In embodiments, read through transcription of the leaky codon occurs from about 1% to about 10% of the time. In embodiments, when read through transcription does not occur the immunoglobulin is expressed in its endogenous format, and the transmembrane reporter cassette is not expressed.

In embodiments, the linker is a peptide linker, e.g. a chain of amino acids from 2 to 24 residues in length. In embodiments, the peptide linker is a dipeptide linker. In embodiments, the linker is a tripeptide linker. In embodiments, the linker is four amino acids in length. In embodiments, the linker comprises Leu-Gly. In embodiments, the linker comprises Gly-Ser-Gly. In embodiments, the linker comprises Leu-Gly-Ser-Gly. In embodiments, the linker comprises about 4, about 5, about 6, about 7, about 8, about 9 or about 10 amino acid residues. In embodiments, the peptide linker comprises from 4 to 24 amino acid residues. In embodiments, the peptide linker comprises from 5 to 20 amino acid residues. In embodiments, the peptide linker comprises from 7 to 15 amino acid residues.

In embodiments, the leader sequence further comprises a secretory signal peptide. In embodiments, the secretory signal peptide is an IL-2 leader sequence. In embodiments, the secretory signal peptide is a human OSM, VSV-G mouse Ig Kappa, Human IgG2 H, BM40, Secrecon, human IgKVIII, CD33, tPA, human chymotrypsinogen, human trypsinogen-2, human IL-2 or a human serum albumin signal peptide. In embodiments, the secretory signal peptide comprises the IL-2 leader sequence MYRMQLLSCIALSLALVINS (SEQ ID NO: 2). Those skilled in the art will understand that signal peptides can be predicted using algorithms known in the art, e.g. the SignalP-5.0 Server at/www.cbs.dtu.dk/services/SignalP/and the SecretomeP 2.0 Server at www.cbs.dtu.dk/services/SecretomeP/.

The affinity tag of the nucleic acid construct can be used for later isolation or purification of a protein expressed by the construct. In embodiments, the affinity tag comprises a StrepII, hexahistadine, FLAG, HA, Myc, VA, GST, beta-GAL, MBP or VSV-G tag. In embodiments, the affinity tag comprises from about 1 to about 18 tandem repeats of a tag. In embodiments, the affinity tag comprises from about 2 to about 15 tandem repeats of a tag. In embodiments, the affinity tag comprises from about 3 to about 10 tandem repeats of a tag. In embodiments, the affinity tag comprises 3 tandem repeats of a tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a tag.

In embodiments, the affinity tag comprises a StrepII-tag. In embodiments, the affinity tag comprises tandem repeats of a StrepII-tag. In embodiments, the affinity tag comprises from about 1 to about 18 tandem repeats of a StrepII-tag. In embodiments, the affinity tag comprises from about 2 to about 15 tandem repeats of a StrepII-tag. In embodiments, the affinity tag comprises from about 3 to about 10 tandem repeats of a StrepII-tag. In embodiments, the affinity tag comprises 3 tandem repeats of a StrepII-tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a StrepII-tag. In embodiments, the tandem repeats have a tag linker in between repeats. In embodiments, the tag linker is a dipeptide or a tripeptide. In embodiments, the tag linker is the dipeptide Ser-Ala. In embodiments, the tag linker comprises the (G4S) 2 linker (GGGGSGGGGS; SEQ ID NO: 8). In embodiments, the tag linker is the (G4S) 2 linker (GGGGSGGGGS; SEQ ID NO: 8).

In embodiments, the StrepII-tag comprises an eight amino acid peptide sequence of WSHPQFEK (SEQ ID NO: 1). In embodiments, the affinity tag comprises WSHPQFEKSAWSHPQFEKSAWSHPQFEK (SEQ ID NO: 3).

The transmembrane domain encoded by the transmembrane reporter cassette allows the affinity tag to be presented on the surface of cells expressing the nucleic acid construct. In embodiments, the transmembrane domain comprises a hydrophobic α-helix. In embodiments, the transmembrane domain is an IgG transmembrane domain. In embodiments, the transmembrane domain is a human IgG1 transmembrane domain. In embodiments, the transmembrane domain includes a mouse IgG transmembrane domain. In embodiments, the transmembrane domain includes a mouse IgG1, IgG2a, IgG2b or IgG2c transmembrane domain. In embodiments, the transmembrane domain is the transmembrane domain of the mouse proteins Tmem53, Lrtm1 or Nrg1.

The fluorescent reporter protein allows detection of cells expressing the nucleic acid construct. In embodiments, the fluorescent reporter protein comprises a green fluorescent protein, a yellow fluorescent protein, a cyan fluorescent protein, a red fluorescent protein, a blue fluorescent protein, a red fluorescent protein or an orange fluorescent protein. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP). In embodiments, the fluorescent reporter protein comprises red fluorescent protein (RFP). In embodiments, the red fluorescent protein is monomeric cherry (mCherry) or tandem dimer Tomato (tdTomato).

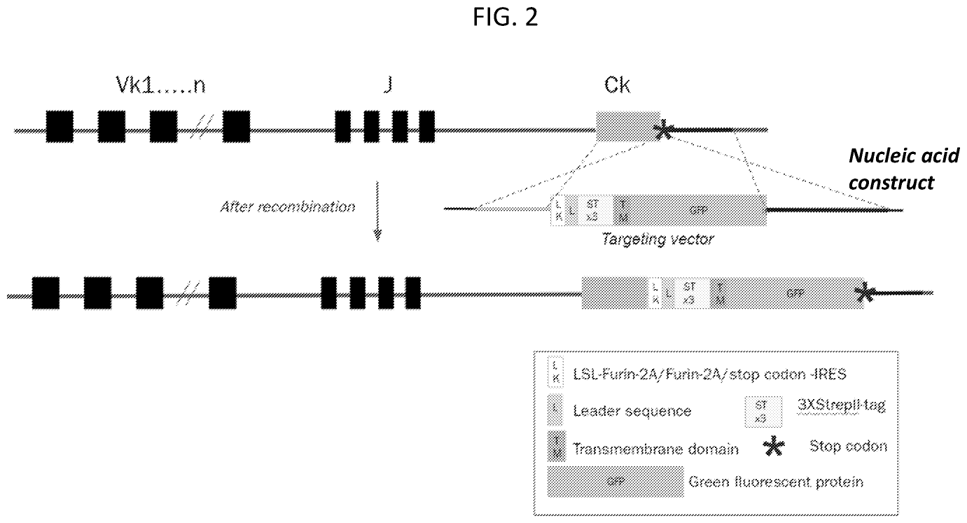

In embodiments, the nucleic acid construct is the nucleic acid construct shown schematically in FIG. 2 for a specific embodiment incorporated into the light chain kappa constant region, where the black rectangles represent the V and J segments of the region; LK represents a linker sequence; L represents a leader sequence, STX3 represents a three tandem repeats of the Strep-II tag, TM represents a transmembrane domain, and GFP represents a green fluorescent protein reporter. In other embodiments (not shown), the nucleic acid construct is incorporated into the light chain lambda constant region or the heavy chain constant region.

### M. Methods of Generating Immunoglobulin Reporter Modified Cells and Organisms

In embodiments, provided herein is a method of generating a genetically modified non-human mammalian cell, the method comprising:
(a) introducing an immunoglobulin reporter nucleic acid construct described herein into the non-human mammal cell; and
(b) introducing a nuclease into the non-human mammal cell, wherein the nuclease causes a single strand break or a double strand break at an immunoglobulin constant domain locus in a genome of the non-human mammal cell, and the nucleic acid construct is integrated into the genome of the non-human mammal cell at the immunoglobulin constant domain locus by homologous recombination.

In embodiments, the nuclease causes a double strand break. In embodiments, the nuclease causes a single strand break (e.g., a nick).

The nuclease may be introduced into the cell using methods known in the art. In embodiments, introducing the nuclease comprises introducing an expression construct encoding the nuclease. In embodiments, the introducing of the expression construct is via injection or electroporation. In embodiments, introducing the nuclease comprises introducing a plasmid encoding the nuclease. In embodiments, introducing the nuclease comprises introducing a viral vector encoding the nuclease. In embodiments, introducing the nuclease comprises introducing a mRNA encoding the nuclease. In embodiments, the mRNA comprises one or more modified bases. In embodiments, the mRNA is encapsulated in a lipid nanoparticle. In embodiments, the introducing of the plasmid, viral vector or mRNA is via injection or electroporation. In embodiments, introducing the nuclease comprises introducing the nuclease protein directly into the cell. In embodiments, the introducing of the nuclease protein directly into the cell is via injection or electroporation.

In embodiments, the immunoglobulin constant domain locus is an immunoglobulin light chain constant domain locus. In embodiments, the immunoglobulin light chain constant domain locus is an immunoglobulin kappa constant domain locus. In embodiments, the immunoglobulin light chain constant domain locus is an immunoglobulin lambda constant domain locus. In one aspect, the immunoglobulin constant domain locus is an immunoglobulin heavy chain constant domain locus.

In embodiments, the immunoglobulin constant domain locus is an immunoglobulin heavy chain constant domain locus. In embodiments, the immunoglobulin heavy chain constant domain locus is a gamma, delta, alpha, mu or epsilon immunoglobulin constant domain locus.

In embodiments, the nuclease is a nuclease as described herein. In embodiments, the nuclease comprises a Zinc Finger nuclease (ZFN), a transcription activator-Like Effector Nuclease (TALEN), a Meganuclease, or a Clustered Regularly Interspaced Short Palindromic Repeats (CRISPR)-associated (Cas) protein and a guide RNA (gRNA). In embodiments, the gRNA comprises a CRISPR RNA (crRNA) that targets a recognition site and a trans-activating CRISPR RNA (tracrRNA). In embodiments, the CRISPR-Cas protein comprises Cas9. In embodiments, the Cas protein includes Cas9, Cas12 Cas12a, Cas13, Cas14 or CasΦ. In embodiments, the CRISPR-Cas protein comprises Cas3, Cas8, Cas10, Cas11, Cas 12, Cas12a, Cas13, Cas14 or CasΦ.

In embodiments, the non-human mammal cell is from a mammal used in scientific research. In embodiments, the non-human mammal cell is a rodent cell. In embodiments, the rodent cell is a rat cell or a mouse cell.

In embodiments, the non-human mammal cell is a pluripotent cell. In embodiments, the pluripotent cell is a non-human zygote. In embodiments, the pluripotent cell is a mouse zygote. In embodiments, the pluripotent cell is a rat zygote. In embodiments, the pluripotent cell is a non-human embryonic stem (ES) cell. In embodiments, the pluripotent cell is a mouse embryonic stem (ES) cell or rat embryonic stem (ES) cell.

In embodiments, the zygote is injected with a nucleic acid construct described herein. In embodiments, the nucleic acid construct is injected into a pronucleus of the zygote. In embodiments, the microinjected zygote is implanted in the oviduct of a pseudo-pregnant female rodent. In embodiments, the pseudo-pregnant female rodent is a mouse. In embodiments, the pseudo-pregnant female rodent is a rat. In embodiments, the implanted zygote develops into a fetus and is birthed to provide a genetically modified non-human mammal. In embodiments, the genetically modified non-human mammal is a mouse. In embodiments, the genetically modified non-human mammal is a rat.

In embodiments, the method of generating a genetically modified non-human mammal cell further comprises isolating the genetically modified non-human mammal cell in which the nucleic acid construct is integrated at an immunoglobulin constant domain locus.

In embodiments, also provided herein is a genetically modified a non-human mammal cell generated by the above method of generating a genetically modified non-human mammal cell. In embodiments, the non-human mammal is a rodent.

In embodiments of the method of generating a genetically modified non-human mammal cell, the method further comprises steps for generating a transgenic non-human mammal. In embodiments, the method further comprises injecting the genetic editing materials into a zygote or the engineered isolated cell into a blastocyst and generating a transgenic non-human mammal comprising the nucleic acid construct integrated at an immunoglobulin constant domain locus.

In embodiments, the disclosure provides a genetically modified non-human transgenic mammal generated by this method. In embodiments, the transgenic mammal is a rodent. In embodiments, the rodent is a rat or a mouse.

### N. Immunoglobulin Reporter Modified Cells

In embodiments, provided herein is a genetically modified non-human mammal cell comprising a genome comprising an immunoglobulin reporter nucleic acid construct described herein integrated into an immunoglobulin constant domain locus.

In embodiments of the cell, the immunoglobulin constant domain locus is an immunoglobulin light chain constant domain locus. In embodiments, the immunoglobulin light chain constant domain locus is an immunoglobulin kappa constant domain locus. In embodiments, the immunoglobulin light chain constant domain locus is an immunoglobulin lambda constant domain locus. In embodiments, the immunoglobulin constant domain locus is an immunoglobulin heavy chain constant domain locus.

In embodiments of the cell, the immunoglobulin constant domain locus is an immunoglobulin heavy chain constant domain locus. In embodiments, the immunoglobulin heavy chain constant domain locus is a gamma, delta, alpha, mu or epsilon immunoglobulin constant domain locus.

In embodiments, the immunoglobulin expressing cell is obtained from an immunized mammal. In embodiments, the immunized mammal is a rodent. In embodiments, the immunized mammal is a mouse or a rat.

In embodiments, the cell is an immunoglobulin expressing cell. In embodiments, the immunoglobulin expressing cell is an immature B cells or a descendant of an immature B cell. In embodiments, the cell is a hybridoma, a stem cell or an immortalized cell. In embodiments, the stem cell is an embryonic stem cell. In embodiments, the stem cell is an adult stem cell. In embodiments, the stem cell is an induced pluripotent stem cell. In embodiments, the stem cell is a perinatal stem cell.

In embodiments, the genetically modified non-human mammal cell expresses an immunoglobulin kappa light chain. In embodiments, the genetically modified non-human mammal cell expresses an immunoglobulin lambda light chain. In embodiments, the genetically modified non-human mammal cell expresses an immunoglobulin heavy chain.

In embodiments, the genetically modified non-human mammal cell expresses a fusion protein comprising an affinity tag, a transmembrane domain and a fluorescent reporter protein. In embodiments, the affinity tag is expressed on a cell surface of the non-human mammal cell. Examples of affinity tags that can be expressed on the cell surface of the non-human mammal cell are described herein.

In embodiments, the affinity tag comprises a StrepII, hexahistadine, FLAG, HA, Myc,

VA, GST, beta-GAL, MBP or VSV-G tag. In embodiments, the affinity tag comprises from about 1 to about 18 tandem repeats of a tag. In embodiments, the affinity tag comprises from about 2 to about 15 tandem repeats of a tag. In embodiments, the affinity tag comprises from about 3 to about 10 tandem repeats of a tag. In embodiments, the affinity tag comprises 3 tandem repeats of a tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a tag.

In embodiments, the affinity tag comprises a StrepII-tag. In embodiments, the affinity tag comprises tandem repeats of a StrepII-tag. In embodiments, the affinity tag comprises from about 1 to about 18 tandem repeats of a StrepII-tag. In embodiments, the affinity tag comprises from about 2 to about 15 tandem repeats of a StrepII-tag. In embodiments, the affinity tag comprises from about 3 to about 10 tandem repeats of a StrepII-tag. In embodiments, the affinity tag comprises 3 tandem repeats of a StrepII-tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a StrepII-tag. In embodiments, the tandem repeats have a tag linker in between repeats. In embodiments, the tag linker is a dipeptide or a tripeptide. In embodiments, the tag linker is the dipeptide Ser-Ala.

In embodiments, the StrepII-tag comprises an eight amino acid peptide sequence of WSHPQFEK (SEQ ID NO: 1). In embodiments, the affinity tag comprises WSHPQFEKSAWSHPQFEKSAWSHPQFEK (SEQ ID NO: 3).

In embodiments, the fluorescent reporter protein is exposed on a cytosolic surface of the non-human mammal cell.

In embodiments, the fluorescent reporter protein comprises a green fluorescent protein, a yellow fluorescent protein, a cyan fluorescent protein, a red fluorescent protein, a blue fluorescent protein, a red fluorescent protein or an orange fluorescent protein. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP). In embodiments, the fluorescent reporter protein comprises red fluorescent protein (RFP). In embodiments, the red fluorescent protein is monomeric cherry (mCherry) or tandem dimer Tomato (tdTomato).

In embodiments of the cell, expression of the fusion protein is driven by an endogenous immunoglobulin transcription regulator. In embodiments, the endogenous immunoglobulin transcription regulator is an endogenous immunoglobulin light chain transcription regulator. In embodiments, the endogenous immunoglobulin light chain transcription regulator comprises a promoter, and other cis-regulatory elements in the mouse light chain locus. In embodiments, the endogenous immunoglobulin transcription regulator is an endogenous immunoglobulin heavy chain transcription regulator. In embodiments, the endogenous immunoglobulin heavy chain transcription regulator comprises a promoter, and other cis-regulatory elements in the mouse heavy chain locus.

### O. Methods for Identifying Immunoglobulin Reporter Modified Cells

In embodiments, provided herein is a method for identifying immunoglobulin expressing cells obtained from a genetically modified immunoglobulin reporter non-human mammal, the method comprising:
(a) obtaining cells from a genetically modified immunoglobulin reporter non-human mammal described herein;
(b) screening the cells obtained from the genetically modified non-human mammal for expression of a fusion protein comprising an affinity tag, a transmembrane domain and a fluorescent reporter protein; and
(c) identifying immunoglobulin expressing cells based on expression of the fusion protein.

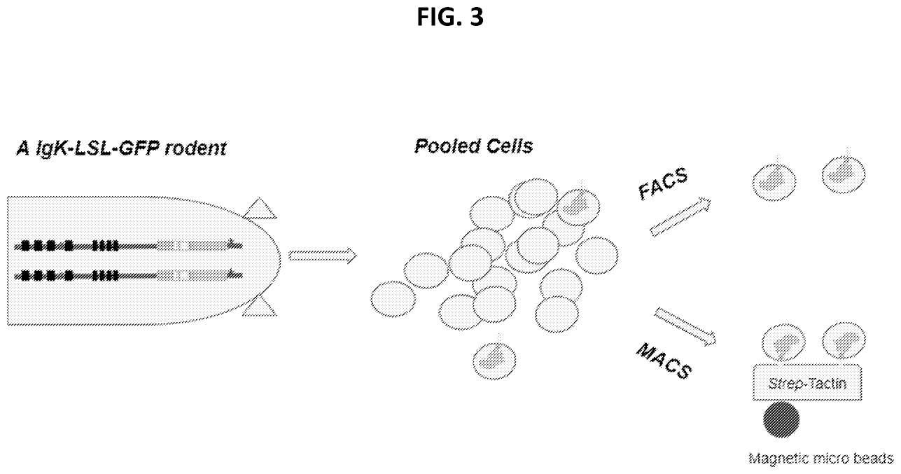

In embodiments, of the method for isolating cells, the cells are screened by fluorescent activated cell sorting (FACS) or magnetic activated cell sorting (MACS). In embodiments, of the method for isolating cells, the cells are screened by fluorescent activated cell sorting (FACS). In embodiments, of the method for isolating cells, the cells are screened by magnetic activated cell sorting (MACS). The techniques of both FACS and MACS are known in the art and are described elsewhere herein. In embodiments, an example process of obtaining cells from an immunoglobulin reporter modified rodent, pooling the cells and separating the cells using either FACS or MACS is shown schematically in FIG. 3. In embodiments of the methods of isolating cells, the cells are separated using the affinity tag expressed on the surfaces of the cells, as described herein. In embodiments where the cells are separated using the affinity tag, the cells are separated using an affinity column or an affinity resin that binds the affinity tag using methods known in the art.

In embodiments, the affinity tag is expressed on a cell surface of the genetically modified non-human mammal cell.

In embodiments, the affinity tag comprises a StrepII, hexahistadine, FLAG, HA, Myc, VA, GST, beta-GAL, MBP or VSV-G tag. In embodiments, the affinity tag comprises from about 1 to about 18 tandem repeats of a tag. In embodiments, the affinity tag comprises from about 2 to about 15 tandem repeats of a tag. In embodiments, the affinity tag comprises from about 3 to about 10 tandem repeats of a tag. In embodiments, the affinity tag comprises 3 tandem repeats of a tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a tag.

In embodiments, the affinity tag comprises a StrepII-tag. In embodiments, the affinity tag comprises tandem repeats of a StrepII-tag. In embodiments, the affinity tag comprises from about 1 to about 18 tandem repeats of a StrepII-tag. In embodiments, the affinity tag comprises from about 2 to about 15 tandem repeats of a StrepII-tag. In embodiments, the affinity tag comprises from about 3 to about 10 tandem repeats of a StrepII-tag. In embodiments, the affinity tag comprises 3 tandem repeats of a StrepII-tag. In embodiments, the affinity tag comprises from about 1, about 2, about 3, about 4, about 5, about 6, about 7, about 8, about 9, about 10, about 11, about 12, about 13, about 14, about 15, about 16, about 17 or about 18 tandem repeats of a StrepII-tag. In embodiments, the tandem repeats have a tag linker in between repeats. In embodiments, the tag linker is a dipeptide or a tripeptide. In embodiments, the tag linker is the dipeptide Ser-Ala.

In embodiments, the StrepII-tag comprises an eight amino acid peptide sequence of WSHPQFEK (SEQ ID NO: 1). In embodiments, the affinity tag comprises WSHPQFEKSAWSHPQFEKSAWSHPQFEK (SEQ ID NO: 3).

In embodiments, the fluorescent reporter protein is exposed on a cytosolic surface of the non-human mammal cell.

In embodiments, the fluorescent reporter protein comprises a green fluorescent protein, a yellow fluorescent protein, a cyan fluorescent protein, a red fluorescent protein, a blue fluorescent protein, a red fluorescent protein or an orange fluorescent protein. In embodiments, the fluorescent reporter protein comprises green fluorescent protein (GFP), enhanced green fluorescent protein (EGFP), enhanced yellow fluorescent protein (EYFP) or enhanced cyan fluorescent protein (ECFP). In embodiments, the fluorescent reporter protein comprises red fluorescent protein (RFP). In embodiments, the red fluorescent protein is monomeric cherry (mCherry) or tandem dimer Tomato (tdTomato).

In embodiments of the method, the genetically modified non-human mammal has been immunized with an antigen of interest. In embodiments, the immunoglobulin expressing cells express an immunoglobulin kappa light chain. In embodiments, the immunoglobulin expressing cells express an immunoglobulin lambda light chain. In embodiments, the immunoglobulin expressing cells express an immunoglobulin heavy chain. In embodiments, the immunoglobulin expressing cells comprise immature B cells and their descendants.

In embodiments, the method further comprises isolating an immunoglobulin expressed from the cell obtained from a genetically modified non-human mammal. In embodiments, provided herein is an immunoglobulin obtained by this method.

In embodiments, provided herein is a method of producing a therapeutic or diagnostic immunoglobulin, the method comprising:
(i) cloning a variable domain of an immunoglobulin disclosed herein; and
(ii) generating the therapeutic or diagnostic immunoglobulin comprising the variable domain obtained in (i).

In embodiments, also provided herein is a method of producing a monoclonal antibody, the method comprising:
(i) obtaining immunoglobulin expressing cells from a genetically modified non-human mammal disclosed herein;
(ii) immortalizing the immunoglobulin expressing cells obtained in (i); and
(iii) isolating monoclonal antibodies expressed by the immortalized immunoglobulin expressing cells, or nucleic acid sequences encoding the monoclonal antibodies.

In embodiments, this method further comprises:
(iv) cloning a variable domain of the isolated monoclonal antibody; and
(v) producing a therapeutic or diagnostic antibody comprising the cloned variable domain.

In embodiments, provided herein are a therapeutic or diagnostic antibody produced by the above methods.

### P. Incorporation by Reference

All references cited herein, including patents, patent applications, papers, text books and the like, and the references cited therein, to the extent that they are not already, are hereby incorporated herein by reference in their entirety.

### WORKING EXAMPLES

### Example 1. Construction of Immunoglobulin Labeling Cassette and Targeting Vector

Step 1: The transmembrane labelling cassette is assembled by ligating the component sequences to form a contiguous cassette. In this Example, the labelling cassette includes sequences encoding a linker, leader sequence, three repeats of the Strep-II tag with tag linkers (WSHPQFEKSAWSHPQFEKSAWSHPQFEK ((SEQ ID NO: 3), a transmembrane domain, and a green fluorescent protein (GFP) as shown schematically in FIG. 2.

The three different linker options are:

Option 1: LSL-Furin-2A. This linker comprises a leaky stop codon, a Leu-Gly linker sequence, a Furin protease recognition and cleavage site, and 2A self-cleavage peptide. The advantage of this design is to maintain the majority of the IgK in its endogenous format. Since the leaky stop codon only allows ˜5% transcription read through, the expression level of StrepII-tag-GFP would be only 5% of total IgK. Therefore, it is possible for this design may be that the StrepII-tag-GFP will be expressed at too low of a level to effectively enrich such cells using FACS/MACS in some instances.

Option 2: Furin-2A. This linker comprises a Furin protease recognition and cleavage site and a 2A self-cleavage peptide. The advantage of this linker is that it ensures high expression of StrepII-tag-GFP, but this level of expression may be toxic to the cells in some instances.

Option 3: stop codon-IRES (Internal Ribosomal Entry Site). This linker comprises a stop codon followed by an IRES, which allows the reporter gene to be transcribed as a separate protein from the immunoglobulin. The IRES offers StrepII-tag-GFP expression levels between the above two strategies.

Step 2: The construct from step 1 is ligated into a targeting vector with homologous flanking regions targeting the stop codon of the rodent Ig kappa (IgK) genomic region. Vectors that can be used for knock-in include pUC18, pUC19 and pBluescript II KS+. This vector will knock-in the StrepII-tag-GFP labeling cassette at the stop codon of IgK as shown schematically in FIG. 2.

Alternatively, synthetic single-stranded DNA can be synthesized with a 200-500 bp homology defining region on each side of the labeling cassette to form a synthetic targeting cassette. This synthetic targeting cassette construct can be incorporated directly using CRISPR targeting systems.

### Example 2. In Vitro Evaluation of Strep II-Tag and GFP Expression Levels

To ensure expression levels of StrepII-tag-GFP that will be compatible for downstream application, in vitro experiments are performed with targeting vectors using rodent B cell lines to evaluate StrepII-tag and GFP expression levels alongside IgK expression levels. The vector that gives the highest IgK expression level as well as decent StrepII-tag and GFP levels for downstream applications will be chosen for further study. Antibodies secreted will be quantified via biochemical measurement, such as Octet. Antibodies displayed on the cell surface will be detected via flow analysis. Briefly, cells will be incubated with fluorescent labeled anti Immunoglobulin antibodies at 4 degrees C. for 30 min; the fluorescent signals will be measured using a flow cytometer. To ensure that the expression of the labelling cassette at the IgK locus does not interfere with IgK function, in vitro tests using rodent B-cell lines will be conducted to identify ideal linker sequences.

### Example 3. Generation of IgK Reporter Rodent Line

Mouse embryonic stem cells are transformed with the targeting vector to enable insertion of the labelling cassette at the IgK stop codon via homologous recombination. To enhance the efficiency of this targeted knock-in at the IgK stop codon, the CRISPR/Cas9 system will be used. SgRNA components targeting the adjacent region around the IgK stop codon in the genome are designed and synthesized, followed by assembly with the Cas9 enzyme to form the RNP complex and co-injection with the homologous recombination repair (HDR) template as a single stranded DNA or a vector into a fertilized oocyte or Embryonic stem cells. Homologous recombination will enable the donor fragment, which contains the labeling cassette, to integrate into the locus after the targeted double strand break caused by Cas9. Embryonic stem cells that undergo successful recombination (as determined by southern blot analysis and PCR) are microinjected into blastocysts to generate transgenic mice.

mAb expressing cells are obtained from transgenic mice and the labelling efficiency is evaluated. Briefly, the antibody expressing cells will be isolated using traditional flow markers via FACS. Cells will be incubated with fluorescent labeled anti Immunoglobulin antibodies at 4 degrees C. for 30 min. The fluorescent signals will be measured using a flow cytometer.

### Example 4. Construction of Conditional Reporter Labeling Cassette and Targeting Vector

Step 1: The transmembrane labelling cassette transgene is assembled by ligating the component sequences to form a contiguous cassette. The labelling cassette includes a CAG promoter, a leader sequence, the LoxP-Stop-LoxP cassette, three tandem repeats of the Strep-II tag, a transmembrane domain, and a green fluorescent protein (GFP) as shown schematically in FIG. 1A.

Step 2: The construct from step 1 is ligated into a targeting vector with homologous flanking regions targeting intron 1 of the ROSA26 genomic region. A splice acceptor (SA) sequence followed by the DNA cassette is inserted into an Xba1 restriction site within the first intron of the ROSA26 gene. This vector will knock-in the StrepII-tag-GFP labeling cassette in the safe harbor ROSA26 locus as shown schematically in FIG. 1A.

Alternatively, synthetic single-stranded DNA can be synthesized with a 200-500 bp homology defining region on each side of the labeling cassette to form a synthetic targeting cassette. This synthetic targeting cassette construct can be incorporated directly into intron 1 of ROSA26 using CRISPR targeting systems.

### Example 5. Generation of Conditional Reporter Rodent Line

Mouse embryonic stem cells are transformed with the transgene targeting vector to enable insertion of the labelling cassette at intron 1 of ROSA26 via homologous recombination. As an alternative, the CRISPR/Cas9 system is a targeted knock-in at intron 1 of ROSA26. SgRNA components targeting at intron 1 of ROSA26 in the genome are designed and synthesized, followed by assembly with the Cas9 enzyme to form the RNP complex and co-injection or electroporation with the homologous recombination repair (HDR) template as a single stranded DNA or a vector into a fertilized oocyte or Embryonic stem cells. Homologous recombination will enable the donor fragment, which contains the labeling cassette, to integrate into the locus after the targeted double strand break caused by Cas9. Embryonic stem cells that undergo successful recombination (as determined by southern blot analysis and PCR) are microinjected into blastocysts to generate transgenic mice.

Mice containing the integrated transgene will be crossed with a Cre switch line mouse, a mouse line having Cre under control of the tissue specific promoter of interest. In one experiment, the Cre switch line mouse is a Blimp1-CreERT2 mouse line, which expresses Cre under control of the Blimp1 promoter that is expressed in plasmablasts and plasma cells. In another embodiment, the switch line mouse is a JchaincreERT2 mouse.

### Example 6. Generation of a Reporter Rodent Line from a Zygote

A vector as described in Example 1 or 3 is injected directly into a mouse zygote. The vector is microinjected into pronuclei of zygotes (fertilized mouse oocytes). The resultant embryos are implanted in the oviducts of pseudopregnant females and allowed to develop to term. The embryos are expelled into the oviduct of the mice and the wound is closed with wound clips. Mice are examined on days 18-21 for the delivery of live offspring. Newborn mice are analyzed for expression of the transmembrane labeling construct using methods described above.

SEQUENCES

| | | SEQ |
|---|---|---|
| Sequence | | ID |
| Identification | | NO: |
| Strep-II tag | WSHPQFEK | 1 |
| IL-2 leader | MYRMQLLSCIALSLALVINS | 2 |
| sequence | | |
| Step-II affinity | WSHPQFEKSAWSHPQFEKSA | 3 |
| tag | WSHPQFEK | |
| T2A peptide | EGRGSLLTCGDVEENPGP | 4 |
| P2A peptide | ATNFSLLKQAGDVEENPGP | 5 |
| E2A peptide | QCTNYALLKLAGDVESNPGP | 6 |
| F2A peptide | VKQTLNFDLLKLAGDVESN | 7 |
| | PGP | |
| (G4S)2 linker | GGGGSGGGGS | 8 |

1. A nucleic acid construct comprising a leader sequence, LoxP-Stop-LoxP cassette, and a transmembrane reporter cassette encoding an affinity tag, a transmembrane (TM) domain and a fluorescent reporter protein.

2. The nucleic acid construct of claim 1, wherein the nucleic acid construct comprises single stranded DNA, double stranded DNA, a plasmid, or a viral vector.

3. The nucleic acid construct of claim 1, further comprising a first homology arm and a second homology arm that are homologous to a first target sequence and a second target sequence, respectively, within a safe harbor locus in a non-human mammal.

4. (canceled)

5. The nucleic acid construct of claim 3, wherein the safe harbor locus comprises a Rosa26 locus on chromosome 6 in a genome of a mouse or a Hipp11 locus on chromosome 11 in a genome of a mouse.

6-8. (canceled)

9. The nucleic acid construct of claim 1, wherein the leader sequence comprises a secretory signal peptide.

10. The nucleic acid construct of claim 9, wherein the secretory signal peptide comprises the IL-2 leader sequence MYRMQLLSCIALSLALVINS (SEQ ID NO:2).

11. The nucleic acid construct of claim 1, wherein the affinity tag comprises a StrepII-tag.

12-17. (canceled)

18. A method of generating a genetically modified non-human mammal cell, the method comprising:
(a) introducing the nucleic acid construct according to claim 1 into the non-human mammal cell; and
(b) introducing a nuclease into the non-human mammal cell, wherein the nuclease causes a single strand break or a double strand break at a safe harbor locus in a genome of the non-human mammal cell, wherein the nucleic acid construct is integrated into the genome of the non-human mammal cell at the safe harbor locus by homologous recombination.

19. The method of claim 18, wherein introducing the nuclease comprises introducing an expression construct encoding the nuclease.

20. (canceled)

21. The method of claim 18, wherein the nuclease comprises a Zinc Finger nuclease (ZFN), a transcription activator-Like Effector Nuclease (TALEN), a Meganuclease, or a Clustered Regularly Interspaced Short Palindromic Repeats (CRISPR)-associated (Cas) protein and a guide RNA (gRNA).

22-39. (canceled)

40. A genetically modified non-human mammal with cells that express a fusion protein comprising an affinity tag, a transmembrane domain and a fluorescent reporter protein generated by the method of claim 18.

41. A genetically modified non-human mammal cell comprising a genome comprising the nucleic acid construct of claim 1 integrated into a safe harbor locus.

42-48. (canceled)

49. A method for isolating cells obtained from a genetically modified non-human mammal, the method comprising:
(a) obtaining cells from a genetically modified non-human mammal of claim 40;
(b) screening the cells obtained from the genetically modified non-human mammal for expression of a fusion protein comprising an affinity tag, a transmembrane domain and a fluorescent reporter protein; and
(c) isolating cells expressing the fusion protein.

50-54. (canceled)

55. A nucleic acid construct comprising a linker, a leader sequence, and a transmembrane reporter cassette encoding an affinity tag, a transmembrane domain and a fluorescent reporter.

56-64. (canceled)

65. The nucleic acid construct of claim 55, wherein the linker comprises: a) a stop codon and an Internal Ribosomal Entry Site (IRES); b) a protease recognition site and a self-cleaving peptide; or c) a leaky stop codon (LSC) with a peptide linker, a protease recognition site, and a self-cleaving peptide.

66-67. (canceled)

68. The nucleic acid construct of claim 65, wherein the protease recognition site comprises a Furin protease recognition site.

69-83. (canceled)

84. A method of generating a genetically modified non-human mammalian cell, the method comprising:
(a) introducing a nucleic acid construct according to claim 55 into the non-human mammal cell; and
(b) introducing a nuclease into the non-human mammal cell, wherein the nuclease causes a single strand break or a double strand break at an immunoglobulin constant domain locus in a genome of the non-human mammal cell, and the nucleic acid construct is integrated into the genome of the non-human mammal cell at the immunoglobulin constant domain locus by homologous recombination.

85-100. (canceled)

101. A genetically modified non-human mammal or a genetically modified non-human mammal cell generated by the method of claim 84.

102. (canceled)

103. (canceled)

104. A genetically modified non-human mammal cell comprising a genome comprising a nucleic acid construct of claim 55 integrated into an immunoglobulin constant domain locus.

105-124. (canceled)

125. A method for identifying immunoglobulin expressing cells obtained from a genetically modified non-human mammal, the method comprising:
(a) obtaining cells from a genetically modified non-human mammal of claim 101;
(b) screening the cells obtained from the genetically modified non-human mammal for expression of a fusion protein comprising an affinity tag, a transmembrane domain and a fluorescent reporter protein; and
(c) identifying immunoglobulin expressing cells based on expression of the fusion protein.

126-141. (canceled)

142. A method of producing a therapeutic or diagnostic immunoglobulin, the method comprising:
(i) isolating an immunoglobulin expressed from the cells of claim 125;
(ii) cloning a variable domain of the immunoglobulin in (i); and
(iii) generating the therapeutic or diagnostic immunoglobulin comprising the variable domain obtained in (ii).

143. A method of producing a monoclonal antibody, the method comprising:
(i) obtaining immunoglobulin expressing cells from the genetically modified non-human mammal of claim 101;
(ii) immortalizing the immunoglobulin expressing cells obtained in (i); and
iii) isolating monoclonal antibodies expressed by the immortalized immunoglobulin expressing cells, or nucleic acid sequences encoding the monoclonal antibodies.

144. (canceled)

145. (canceled)


SEQUENCE LISTING (8 sequences)

SEQ ID NO: 1 - protein, 8 aa, synthetic construct
WSHPQFEK

SEQ ID NO: 2 - protein, 20 aa, synthetic construct
MYRMQLLSCIALSLALVTNS

SEQ ID NO: 3 - protein, 28 aa, synthetic construct
WSHPQFEKSAWSHPQFEKSAWSHPQFEK

SEQ ID NO: 4 - protein, 18 aa, synthetic construct
EGRGSLLTCGDVEENPGP

SEQ ID NO: 5 - protein, 19 aa, synthetic construct
ATNFSLLKQAGDVEENPGP

SEQ ID NO: 6 - protein, 20 aa, synthetic construct
QCTNYALLKLAGDVESNPGP

SEQ ID NO: 7 - protein, 22 aa, synthetic construct
VKQTLNFDLLKLAGDVESNPGP

SEQ ID NO: 8 - protein, 10 aa, synthetic construct
GGGGSGGGGS